(19) United States
(12) Patent Application Publication
(10) Pub. No.: US 2021/0345897 A1
(43) Pub. Date: Nov. 11, 2021

(54) Heart Rate Variability Composite Scoring and Analysis

(71) Applicant: Elite HRV, Inc., Asheville, NC (US)

(72) Inventors: Jason David Moore, Asheville, NC (US); Alyssa Rae Moore, Asheville, NC (US)

(21) Appl. No.: 16/867,629

(22) Filed: May 6, 2020

(51) Int. Cl. A61B 5/024; A61B 5/00; A61B 5/11

(52) U.S. Cl. CPC A61B 5/02405; A61B 5/7435; A61B 5/0024; A61B 5/6831; A61B 5/1118; A61B 5/7207

(57) ABSTRACT

A system and method for Heart Rate Variability (HRV) data collection and analysis is presented to provide more specific guidance that is based on user HRV data, such as customized scores, customized modifications to specific plans or programs, e.g., guided breathing programs, athletic training plans, validations of treatments or events expected to have an impact on HRV readings. The collected HRV data may be used in live biofeedback type applications, such as including HRV scores or composite scores using HRV data as display graphics in streaming services, using HRV scores or composite scores to modify the behavior of systems such as gaming systems, vehicles, content recommendation systems, ad selection systems, etc. The system and method also presents a user display for teams or groups, with HRV related statistics and recommendations available at various levels of granularity (e.g., group or team, sub-group, individual).

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyright rights whatsoever.

### BACKGROUND

Heart Rate Variability (HRV) is determined from heart beat data and represents variability in inter-beat timing. A heart rate monitor or other sensor detects the ECG or the PPG, i.e., a data measure that varies in relation to the heart's contraction and relaxation. From this the peaks of the heart contraction can be derived and plotted against time. This is in turn allows the timing between peaks to be reported as a time (in milliseconds) between peaks.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a view of artifact detection accuracy in terms of the detection of false positive artifact detection consistent with certain embodiments of the present disclosure.

FIG. 2 is a view of artifact detection accuracy in terms of the detection of true positive artifact detection consistent with certain embodiments of the present disclosure.

FIG. 3 is a view of artifact impact on the system consistent with certain embodiments of the present disclosure.

FIG. 4 is a view of the display of HRV statistics for a user post reading consistent with certain embodiments of the present disclosure.

FIG. 5 is a view of the display of the continuation of HRV statistics for a user post reading consistent with certain embodiments of the present disclosure.

FIG. 6 is a view of the display of the data integration connections for the user device consistent with certain embodiments of the present disclosure.

FIG. 7 is a view of the display of the historical log for a user consistent with certain embodiments of the present disclosure.

FIG. 8 is a view of the historical trends for HRV statistics for a user consistent with certain embodiments of the present disclosure.

FIG. 9 is a view of the connection capability for the sensors associated with the HRV monitoring system consistent with certain embodiments of the present disclosure.

FIG. 10 is a view of the historical trends for HRV statistics for a user related to morning readiness scores and HRV values consistent with certain embodiments of the present disclosure.

FIG. 11 is a view of the detailed data values for HRV statistics for a user related to morning readiness scores and HRV values consistent with certain embodiments of the present disclosure.

FIG. 12 is a view of the informational data for a user related to morning readiness scores and HRV values expressed as autonomic balance consistent with certain embodiments of the present disclosure.

FIG. 13 is a view of the historical trends for HRV statistics for a population related to morning readiness scores and HRV values consistent with certain embodiments of the present disclosure.

FIG. 14 is a view of the historical trends for HRV statistics for a population related to morning readiness scores and HRV values consistent with certain embodiments of the present disclosure.

FIG. 15 is a view of the raw data captured for RR intervals and HRV values consistent with certain embodiments of the present disclosure.

FIG. 16 is a view of the relationship between RR intervals and HRV values consistent with certain embodiments of the present disclosure.

FIG. 17 is a view of the display of HRV statistics and signal quality for a user post reading consistent with certain embodiments of the present disclosure.

FIG. 18 is a view of the user feedback and tagging display consistent with certain embodiments of the present disclosure.

FIG. 19 is a view of the display of HRV statistics and signal quality for a user post reading consistent with certain embodiments of the present disclosure.

FIG. 20 is a view of the composite score reading and data collection process consistent with certain embodiments of the present disclosure.

FIG. 21 is a view of the HRV system configuration consistent with certain embodiments of the present disclosure.

### DETAILED DESCRIPTION

While this disclosure is susceptible of embodiment in many different forms, there is shown in the drawings and will herein be described in detail specific embodiments, with the understanding that the present disclosure of such embodiments is to be considered as an example of the principles and not intended to limit the disclosure to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

Reference throughout this document to “sympathetic”, refers to a part of the nervous system that serves to accelerate the heart rate, constrict blood vessels, and raise blood pressure

Reference throughout this document to “parasympathetic” refers to the portion of the autonomic nervous system that conserves energy as it slows the heart rate, increases intestinal and gland activity, and relaxes sphincter muscles in the gastrointestinal tract.

Reference throughout this document to “HRV” refers to “Heart Rate Variability” which is a measure of the variability in inter-beat timing of a heart as it is actively beating.

Reference throughout this document to “HRV scoring” refers to the development of a score that is calculated utilizing various algorithms to present a scaled score from which comparisons over time may be made.

Reference throughout this document to “Morning Readiness” refers to a scaled score related to a user's particular balance of parasympathetic and sympathetic nervous system activity.

Reference throughout this document to “Composite Scoring” refers to composites scores that have HRV Scoring, Morning Readiness scoring, and/or additional scoring parameters that are not necessarily related to HRV scores.

Reference throughout this document to “Autonomic Balance” refers to changes in a user's Autonomic Nervous System (ANS) as indicated by changes in the user's HRV over time.

Reference throughout this document to a “Readiness Score” refers to a novel readiness score based upon ANS activity changes over time and indicates the user's readiness to tackle life's challenges each day.

The inter-beat intervals or R-R intervals are transmitted to the Elite HRV software wirelessly (currently via Bluetooth) from the finger sensor and used to calculate variability over time in the inter-beat or R-R intervals, i.e., the HRV data. Changes in the inter-beat or R-R intervals are associated with changes in parasympathetic and sympathetic nervous system activity (which influences and can control heart rate, blood pressure, pupil dilation, blood glucose, muscle tension, sexual function, digestion, and energy regulation), and has been used as an indicator of stress levels, inflammation levels, and post-exercise recovery status, among other conditions. As such, it has proven useful in certain areas, such as gauging if an athlete has recovered adequately from a prior workout or is over-trained, estimating cognitive functioning, predicting risk for certain conditions, etc. In fact, HRV data has been correlated to all major causes of death.

The research literature evaluating algorithms for the detection and correction of artifacts in IBI series focuses on data sourced from specific, homogenous subpopulations rather than broad cross-sections. Additionally, source data used in the comparative literature is typically derived from low-noise, research-grade ecg sensors. While this evaluation strategy may suffice for limited clinical contexts in which the population and experimental conditions can be controlled, large-scale consumer HRV applications must satisfy broader requirements. In particular, those consumer applications supporting open compatibility with 3rd party Bluetooth sensors are liable to face significant variance in both the population parameters (e.g. age, athleticism, pathology of users), and the sensor platforms used. Thus, it is insufficient to evaluate artifact detection algorithms using the traditional, narrow source data parameters.

There has been interest in leveraging HRV data (alone or in combination with other data) to make more specific recommendations. For example, competitor HRV4Training offers a suite of estimations geared towards athletes, including the following: Lactate threshold estimation (providing advice on pacing strategies for racing and workouts); Half and full marathon time estimation; VO₂max estimation (cardiorespiratory fitness level); Functional Threshold Power (FTP) estimation (providing advice on pacing strategies for racing and workouts); and Aerobic Endurance (efficiency and cardiac decoupling). Others have used HRV data to generate a variety of scores, e.g., cognitive functioning scores, risk classifications for cardiac events and cardiovascular disease, etc. In general, HRV data has been and continues to be actively researched to determine if HRV data can act as an indicator or biomarker of certain conditions and used for predictions.

A primary use context for HRV data remains athletic training and health predictions. Competitors, such as HRV4Training, have a suite of services geared towards this context. These include team views, calendar views, various customized scores (some calculated on the basis of third-party data in combination with HRV data), and rudimentary predictions (e.g., estimated marathon time).

The primary goal of Heart Rate Variability monitoring and data analysis is to determine how a person's heart rate, between the beats, fluctuates so as to perform analysis on the heart rate data, track heart rate variability over time, create an HRV score, create a “morning readiness score” over time, create composite scores that include additional scoring separate from the HRV scores created, and provide educational courses on Heart Rate Variability.

The HRV system and method herein disclosed describes improved techniques for obtaining usable HRV data using generally available sensors (e.g., physiological sensors, smartphone cameras or other sensors as herein described) and techniques for improving the quality of HRV data or using lower quality HRV data, including improving signal to noise ratios. This will permit more confident HRV scoring when using lower quality sensors such as cameras and permit HRV scoring to be produced from smaller data samples, i.e., reducing the time needed to take useful readings.

Although extensive medical research has been conducted on the various uses for HRV data, e.g., workout recovery and performance or health-related predictions, conventionally HRV data (or various scores) have been used to provide general guidance for end users. An exception is in the clinical or research setting, where HRV data has been collected for single (not longitudinal) snapshots of the user's autonomic nervous system, usually derived with longer readings (10 minutes to hours long) with clinical grade equipment.

The user has the opportunity to tag the HRV data collected during any reading with contextual information. This tagged information doesn't affect the scoring, but it is presented with the HRV based scores to assist the user in understanding the data and how it relates to any of a user's goals. In a non-limiting example, the tag data types may include sleep data, exercise data, mood ratings, questionnaires, custom tags/notes, blood glucose level, body weight, as well as other relevant data to be shared with the user. The user may also link the Elite HRV account with third-party apps and services to automate any contextual data collection and display other types of data alongside the HRV data and Morning Readiness scores.

The HRV system utilizes captured data from one or more HRV readings to calculate an HRV Score, scaled on a 1-100 basis, based on the natural log of the Root Mean Square of Successive Differences (RMSSD) for the HRV data collected. Changes in the HRV Score correlate with changes in: breathing and respiratory patterns; physical stress; recovery from physical stress; physical performance; psychological stress and health; emotion and mood; cognitive performance; immune system function; inflammation, posture and structural health; injury; biological age; general health and wellbeing; resilience and adaptability; risk of disease; morbidity and mortality; motivation and willpower; and digestive stress.

Upon completion of the HRV score calculation, a user may compare the calculated HRV score and other non-proprietary HRV parameters to general population and/or demographic-filtered population data to provide an indication of how the user compares with the general population as a whole or with specific filtered portions of the general population. This comparison may provide a user with some indication as to changes in their HRV values with respect to their own historic values as well as historic values for a given population.

The HRV system may also create a daily expressed score for use in tracking a user's HRV values over time. This daily expressed score is known as a Morning Readiness score. The Morning Readiness score is a scaled score (1-10) that shows the relative balance or imbalance in the user's sympathetic and parasympathetic nervous system. The Morning Readiness score correlates with day-to-day fluctuations in the nervous system for an individual, highlighting to the user when major changes may have occurred in the body, based on the user's own unique individual patterns.

Currently, data must be collected from at least two HRV readings to establish a true baseline and to begin calculating the Morning Readiness score. The Morning Readiness score may be generated through automated pattern recognition applied to the user's HRV scores over time. The pattern recognition is based on research and uses statistical methods such as standard deviation and mean over time to create the Morning Readiness score each day. This pattern recognition is further refined by research in the HRV system's unique database of HRV data collected and stored for each user registered with the HRV system. Machine learning algorithms may be applied as testing and data analysis prove machine learning algorithms to be of equal or greater accuracy than the HRV system's human-generated algorithms. The machine learning algorithms utilized by the HRV system may be trained using the HRV system's database in order to produce an algorithm that automatically detects a user's HRV trend and assigns a morning readiness score.

The HRV system may also create custom algorithms to determine customized scores. Such customized scores may include an inflammation score or other scoring parameters that may utilize HRV collected data in combination with other scoring data, or other parameters that are collected from outside data sources. The HRV system has access to large electronic data stores containing large amounts of collected HRV data from users of the system as well as metadata from HRV collected data. Much of the HRV data is labeled with contextual tags (metadata) and can be reviewed to label appropriate portions of the data as training data. The retained HRV data and metadata may be used to create models that classify HRV data into contextual tag categories, proxies and/or equivalents, or new categories. This data categorization and metadata may permit the HRV system to detect conditions of interest in newly collected HRV data through the use of machine learning algorithms.

In an embodiment, the HRV system may utilize such machine learning algorithms to predict various conditions of interest to the users of the HRV system. Such conditions may include the ability to predict physical conditioning, physical performance levels, stress levels, and other physical conditions affecting one or more users. Additionally, the HRV system may utilize data organized into one or more Poincare plots (a two-dimensional plotting of beat variability) to identify conditions based on the pattern of the plotted data points. Utilizing machine learning algorithms to perform an analysis of such data plots may permit the HRV system to discover new conditions, such as, in a non-limiting example, conditions associated with stress, athletic performance, fatigue, and other conditions affecting the users of the HRV system.

As may be appreciated, many such classifications are possible for various types of athletes, gaming competitors, users interested in general health, etc. These classifications may in turn be reported, for example, as a score associated with which category the HRV data is classed or associated, as a predicted risk or performance indicator. Implementation would largely depend on having access to sufficient training data, which may need to be annotated or categorized for use as training and validation data. However, the HRV system currently collects such data and may continue to collect these types of data in a directed manner. The data collection effort may take the form of prompting users to report goals, race times or event performances, training logs, stress levels, etc., in combination with their HRV data readings. This information, in combination with the collected HRV data and HRV metadata, provides the basis for the calculation and creation of composite scores consisting of HRV information and additional data supplied by users.

There have not been any solutions that offer specific guidance to end users based on HRV data that has been collected. This lack of guidance stems from a lack of certainty in the reliability of input HRV data itself and the inability to leverage reliable outcome data that correlates HRV data with specific plans, courses of action, and outcomes. This results in HRV data being used to generally estimate the user's nervous system state and provide equally general feedback. In a non-limiting example, HRV data has been used to provide daily feedback regarding the body's apparent ability to handle a stressful workout, a binary categorization of a user's risk for a condition, etc. In a non-limiting example, an application for a training plan, such as a triathlon training plan, may guide a user through a series of HRV measurements, scores, and plan steps to customize the training for the user based on his or her actual HRV data. An initial HRV reading is taken, followed by a programmed event (e.g., a workout). Thereafter, the application guides the user to take a subsequent, updated HRV reading. Depending on the change, if any, in HRV data collected during the reading and the HRV score, the plan may be modified. The decision as to how the plan is to be modified may be programmed into the application, e.g., based on HRV data research, learning from the community, etc. At the various points in the plan, the application may provide data feedback related to the HRV score, the HRV scoring trend, contextual feedback, the Morning Readiness scores, or a combination of the foregoing. This allows the user(s) to understand, based on HRV data and other contextual data, the effectiveness of the plan, why it has been modified, etc.

While performance feedback in the form of updated HRV score and HRV readings is quite useful to athletes wishing to determine if they are over-trained and should rest, or have reached a specific performance level (e.g., estimated half marathon time), current uses have limited the usefulness of current applications and overlook many potential uses for HRV data, particularly as applied while the user is actively engaged in a training plan or health or lifestyle improvement application, and should have specific, guided adjustments made to that plan.

In an embodiment, with some modification (e.g., continuous reading for live biofeedback), the performance feedback technique is applicable to a wide variety of possible applications, ranging from near-term or planned applications for guided breathing and meditation, to exercise and fitness plan modification and food sensitivity validation based on HRV scores, and even long term plans to use HRV data in novel contexts. Such additional applications may include modifying the behavior of systems like gaming systems, content recommendation systems, or vehicles using HRV data.

Additionally, HRV data tends to be somewhat difficult to understand. This lack of understanding of what HRV data specifically indicates regarding a user's physical condition has resulted in the use of various scores. While these various scores are quite useful in driving home the meaning of a user's current HRV readings, current existing scores may also serve as a defined endpoint to guidance or advice that could flow from the HRV data.

In an embodiment, the system and method described herein plans to provide an improved set of recommendations that include specific guidance based on HRV data and other physiological, behavioral, and outcome-based data. The improved recommendations may be provided periodically, as part of an ongoing plan, or provided in real-time for live biofeedback. This will allow users to more confidently approach a myriad of tasks that could be improved by monitoring HRV data as well as other aforementioned data and tailoring specific feedback on the basis thereof. This may include provision of various custom scores and directed, goal-oriented applications for individuals or groups.

In an embodiment, the HRV system may expand on the scores or indices that are provided to users by leveraging the proprietary database of collected HRV data. Scores of interest to the HRV system, and by extension to the HRV system users, include a recovery score, an inflammation score, a cognitive function score, a readiness score for specific goals (e.g., triathlon readiness), a health score, a fitness score, a stress index, a “tilt” score (gaming/poker term for being stressed), a self-awareness score, and a glucose/HRV/ketones index. Existing research may be useful in designing algorithms to make these predictions. In a non-limiting example, a cognitive function score may be created based on research indicating HRV scores are related to cognitive capability.

In an embodiment, improvements in HRV data quality when collecting such information at home or in non-clinical environments may be achieved through directed signal analysis and data normalization. While high quality HRV data can be obtained using a biosensor specifically designed for the task, such as a finger sensor or chest ECG strap, collection of high quality HRV data remains cumbersome due to the need for biosensors and somewhat extensive collection times. HRV data quality may be enhanced by improvements in signal analysis and data processing leading to shorter data collection times while using existing sensors for collection of HRV data.

In an embodiment, the system and method herein described may provide improved techniques for obtaining usable HRV data, as well as additional data input by a user or a medical service provider, using generally available sensors and input methods (e.g., smartphone cameras). This will permit more confident HRV scoring and combination scoring when using lower quality sensors such as cameras and permit HRV scoring to be produced from smaller data samples, thus reducing the time needed to take useful readings.

The efficacy of any heart rate variability metric critically depends upon the signal to noise ratio of its source data. In particular, series of so called MI's (inter-beat-intervals i.e. time between consecutive R waves in the QRS complex) are susceptible to contamination by artifacts which if ignored or improperly treated, demonstrably deteriorate the accuracy of estimated (Heart Rate Variability) HRV metrics.

Most sensors which detect heart beats for digital signal processing are one of two types: electrocardiogram (ecg) and photoplethysmography (ppg). Additional sensors currently in development may utilize computer vision systems either with or without computer deep learning techniques to collect HRV data from a user. Ecg functions by placing electrodes on or near the user's' chest. With each beat, the human heart generates variations in skin-surface voltage roughly on the order of 1 millivolt. These variations induce electron movement in the ecg electrodes which are captured in computer memory by analog-to-digital conversion. PPG functions by emitting light of known wavelength and intensity onto the user's skin (usually finger or earlobe) and measuring the light reflected back or transmitted across. Because the arterioles and arteries distend when blood is pumped by each heartbeat, the opacity of the tissue varies with the cardiac cycle.

HRV monitoring and related analytics may be provided through a proprietary finger sensor attached to a user and used for collecting HRV data (photoplethysmogram (PPG) data collected using LEDs), although the mobile app allows users to input HRV data using third-party sensors (e.g., chest strap that collects electrocardiogram (ECG) data and provides inter-beat intervals for calculating HRV).

In an embodiment, the system may utilize the physiological sensor, either an electrocardiogram or a photoplethysmogram, to detect heart beats of a user of the system. Upon collection of the measurements from the physiological sensor, the sensor measurements derive the peak of the heart contraction and report the time, in milliseconds, between peaks. This derived set of measurements defines the interbeat intervals or, as commonly known, the R-R intervals. In a non-limiting example, the physiological sensor may be specified as a finger mounted sensor, although other sensors applied to different parts of a user's body may be equally effective in capturing the sensor measurements.

The HRV system has an interest in removing the need to use just a physiological sensor to collected HRV data by using an integrated sensor such as a wearable device that collects HRV data natively (e.g., Apple Watch) or a smartphone or other computer based camera that facilitates image-based HRV data collection, coupled with other data collection such as user supplied blood pressure and pupil dilation information and device data such as data readings from an accelerometer or other sensors installed within an electronic device such as a smart watch, smartphone, tablet, or other computer based sensors. Use of existing sensors of the user's common hardware (e.g., smartphone, smartwatch, laptop, etc.) will provide an expanded access to users and data. Of these sensors, camera finger-based physiology detection sensors are currently in use for collecting HRV data. These sensor readings may be improved by reducing finger movement via reduced reading times or finger stabilization techniques or a magnetic accessor that attaches to the finger to stabilize it, among other methods for finger stabilization.

In an embodiment, camera, face-based, physiology detection is another mechanism to collect image data that can be used to collect HRV data, and derive HRV scores and other biometric data such as heart rate, blood pressure, oxygen levels, CO₂ levels, glucose, ketones, general awareness or alertness, stress, reflex time, resilience, training or related capacity or capability, or a combination of the foregoing through an analysis of facial characteristics and coloration, where such image analysis may be stored and used collectively over time to discern trends in HRV data values. Use of image/video analysis permits a user to point any camera at his or her face and detect heartrate and HRV, as well as other data such as blood pressure, pupil dilation, etc., some or all of which may be combined into a composite score of which HRV data is only a portion. Likewise, remote cameras can be used to determine this data, such as at a sporting event. In almost real-time, it is possible to detect HRV and other data and use this to provide an HRV score, any of the various composite or customized scores that use HRV data, breathing rates, oxygen levels, blood pressure, pupil dilation, eye movement/blinking rate, emotional state and additional such parameters.

Face-based detection is important because it can be used more naturally during certain activities, such as driving a car, whereas finger-over-phone camera or other sensing is not possible or not preferable.

In addition to any existing method of using image data to determine HRV and related biometric data of interest, the HRV system has the ability to use one or more existing databases of HRV data and HRV metadata to improve the accuracy of image-based HRV scoring and other determinations. In a non-limiting example, the HRV system can utilize high quality HRV data and related HRV scores to learn which images or features are associated with the HRV scores. This may involve the use of one or more machine learning algorithms, for example, to categorize images or image features and associate these categories with particular HRV data and scores.

The R-R intervals may be transmitted to a system server using a wireless protocol such as Bluetooth, although this should not be considered limiting as alternative wireless protocols may be used such as BLE, Wi-Fi, NFC, ZigBee, or other such protocols developed in the future, for storage and analysis. The system may have a plurality of software modules that analyze the data to determine hourly and daily measurements for heart rate variability (HRV) in an individual.

After being digitized, the raw waveform is processed by a beat detection algorithm to determine where true heart beats occurred. For ecg signals, beat detection algorithms take the form of QRS complex detection algorithms. QRS detection may utilize wavelet analysis or some other pattern matching system. Algorithms for beat detection for both ecg and ppg vary across devices and software applications. After being processed at this stage, what remain is a series of inter-beat-intervals (IBIs). An IBI is simply the amount of time (usually milliseconds) between two subsequent beats. A typical value might be 1,000 ms, which would in turn correspond to an instantaneous heart rate of 60 bpm. Given a noise free recording under perfect conditions, the IBIs could be used to directly calculate HRV as they are. As such is rarely the case, it is at this point that artifact detection algorithms should be applied to the IBIs to test for any errors or artifacts that may have entered the signal thus far.

In an embodiment, the HRV system software manages connections to multiple sensors, assisting the user in selecting the appropriate sensor for the current measurement. Upon receipt of R-R intervals from the hardware, the Elite HRV software, in real-time (within a second or two), displays the beat patterns and received data visually to the user for live or real-time biofeedback in the form of calculated heart rate, calculated HRV values, visual charts of heart rate patterns and R-R interval patterns. The Elite HRV software also checks, again in real-time, the received data for accuracy and quality. The data quality checks are based on published research standards (typically done manually by physiologists or research teams), historical population data, and patterns in prior data received within the same reading or session, i.e., beats are analyzed recursively throughout the reading as new beat intervals are received. The HRV system also assists the user visually and algorithmically in identifying when the user's heart rate has stabilized at the beginning of a reading.

Upon completion of the storage of all R-R interval data, the system is operative to apply the Root Mean Square of Successive Differences (RMSSD) calculation to the R-R intervals. The RMSSD analytical method is the industry standard time domain measurement for detecting Autonomic Nervous System (specifically Parasympathetic) activity in short-term measurements, where short term is defined as approximately 5 minutes or less. A natural log (ln) is applied to the RMSSD calculation. RMSSD does not chart in a linear fashion, so it can be difficult to conceptualize the magnitude of changes as it rises and falls. Therefore, it is common practice in the application of RMSSD calculations to apply a natural log to produce a number that behaves in a more linearly distributed fashion.

The ln(RMSSD) is expanded to generate a useful 0 to 100 score. The ln(RMSSD) value typically ranges from 0 to 6.5. Using over 6,000,000 readings from an existing proprietary database, the system may able to sift out anomalous readings and create a much more accurate scale where everyone fits in a 0 to 100 range—even Olympians and elite endurance athletes.

The HRV score may correlate with changes in breathing and respiratory patterns, physical stress, recovery from physical stress, physical performance, Psychological stress and health, emotion and mood, cognitive performance, immune system function, inflammation, posture and structural health, injury, biological age, general health and wellbeing, resilience and adaptability, risk of disease, morbidity and mortality, motivation and willpower, and/or digestive stress. The customized HRV score may be transmitted to a medical practitioner or directly to a user, where the medical practitioner or user may compare the customized HRV score, and other non-proprietary HRV parameters, to population data and/or demographic-filtered population data to provide a basis in comparison to a selected population.

In a non-limiting example, when measuring HRV changes before or after specific events, it is recommended that HRV readings should be taken for at least 60 seconds immediately pre- and post any activity or event. For better accuracy in the HRV readings it is recommended that the user keep the same body position between readings that the user wishes to compare to past or future readings.

In an alternative non-limiting example, HRV readings can gather relevant HRV data in as little as 30 seconds duration or as long as 24 hours. However, for Morning Readiness type readings, it is recommended by the HRV system that the user take a two-minute reading to collect HRV data for that time. This data collection effort should be performed after the user's period of longest sleep. Using guidelines transmitted to users from the HRV system most users will perform a data collection HRV reading of between 60 and 180 seconds in duration. For HRV data collection during meditations or live biofeedback, the HRV system recommends taking data collection readings of between 4 and 20 minutes in duration and repeating as often as required by the user. During this data collection period the user has the option to turn on audio and/or visual cues for guided breathing patterns, mindfulness, and meditations.

Additionally, the system has a mobile application (app) for use in capturing and transmitting information between the user and the HRV monitoring system. The mobile app currently focuses on providing general data (e.g., heart rate), scaled HRV score, and Morning Readiness score coupled to high-level or general feedback based on the HRV data. For example, a Morning Readiness score may be presented as a numeric value and a gauge graphic.

In an embodiment, the software modules in the system server may be active to manage connections to multiple sensors, assisting the user in selecting the appropriate sensor for any desired measurement. Upon receipt of the R-R intervals from the sensor(s), regardless of the sensor utilized, the system software immediately performs a set of functions in real-time, where real-time is specified as an interval of less than two seconds from the receipt of the R-R interval information.

Initially, the system software displays the beat patterns and received measurement data visually to the user for live feedback to the user in the form of calculated heart rate, calculated HRV values, visual charts of heart rate patterns and R-R interval patterns. This feedback to the user is also known as biofeedback. Next, the system software is operative to check the received measurement data for accuracy and quality. In a non-limiting example, data quality checks are based upon published research standards, historical population data, and/or patterns in prior data received within the same “reading” or measurement collection activity. Heartbeats are analyzed recursively throughout the reading as new beat intervals are received.

In an embodiment, readings can be as little as 30 seconds in duration or as long as 24 hours. In enhanced data collection utilizing a Machine Learning algorithm and/or additional data analysis techniques may further shorten the time required for performing and HRV data reading to 10 seconds or shorter. For Morning Readiness type readings, recommendations to the user are to take a reading between 60 and 180 seconds in duration, with the average being approximately 120 seconds (2 minutes) after the period of longest sleep, which is typically a morning reading. For meditation actions or live biofeedback for a user, the recommendation is to take a reading of between 4- and 20-minutes duration, repeating as often as the user desires to foster user actions. The Morning Readiness Score may correlate with day-to-day fluctuations in the nervous system for an individual, highlighting to the user when major changes may have occurred in the body, based on their own unique individual patterns.

In a non-limiting example, the ranges of the Morning Readiness score provide information to the user on whether the user is in a Sympathetic or a Parasympathetic status on that given day. In this example, values in the 1-3 portion of the range are in the red zone of a gauge as represented on a gauge score graphic. This indicates a wide swing in balance either towards the Sympathetic or Parasympathetic side. A wide acute swing in either direction is usually in reaction to a strong acute stressor or reaching a threshold of accumulated stress. Values in the 4-6 range are in the yellow zone. Yellow indicates a similar, but not as drastic, change in relative balance as a red indication. Yellow days are often nothing to worry about in isolation. Values in the 7-10 range are the green zone. Green indicates that your relative balance is very close to the user's norm. A perfect 10 score is achieved when the relative balance is slightly Parasympathetic leaning. This means that if the user normally scores around a 45 on the HRV score, then an HRV score of 46 may produce a relative balance score of 10.

In an embodiment, the sensitivity of the 1-10 relative balance score depends on a user's individual patterns. If the user often fluctuates widely day-to-day, then the user's relative balance gauge will become less sensitive to change. If the user's HRV scores hardly fluctuate at all, the relative balance gauge will become more sensitive to small changes. Additionally, utilizing proprietary data analysis algorithms and machine learning systems the sensitivity to small changes may be increased further permitting greater accuracy for the relative balance score and the reporting of any fluctuations in a user's relative balance gauge.

In an embodiment, the data analysis results in an instant HRV score and a morning readiness score that can be used for spot checks, and can be used as a parameter to be analyzed over time to determine long term HRV measurements for an individual. The instant HRV scores are also accumulated and analyzed over time to help physicians and users in tracking HRV, forming a part of the health tracking data for the user. This instant HRV score is also used by professional and elite athletes to analyze their heart rate variability to optimize performance, and may be used by a coach or an automated software algorithm to also create training plans based upon the athlete's performance as shown by the HRV score. The morning readiness score provides a daily baseline indication for the user. This score is trended and charted over time, to help the user understand how acute, short-term, medium-term, and long-term choices and events impact their score over time.

In an embodiment, HRV data readings may currently be taken utilizing various devices where such devices may, include a mobile device have a network connection capability, such as a smart phone, iPad, tablet, wearable mobile device, laptops and other mobile devices, as well as cameras and sensors incorporated directly into fitness equipment. Data may also be able to be generated by sensors built directly into a mobile device and may not require a connection, wired or wireless, to external sensors. During the reading, the user may also have access to audio and/or visual cues to present guidance on breathing patterns, mindfulness, and meditations.

In an embodiment, after an HRV data reading is completed, the user, whether a medical professional taking a reading from a patient or a user taking a reading from their own body, may have an opportunity to tag the HRV reading with contextual information. The tag information may be attached to the completed score derived from the HRV reading but does not affect the calculation, analysis, or creation of the completed score. An optimized future system may utilize the tagged HRV reading with contextual information to discover meaningful patterns identified in data analysis or by machine learning algorithms to generated composite metrics that utilize the contextual information in the metric generation. The tag information may be attached to the score record to assist the user in understanding the HRV reading and score data and how the data relates to any goals that have been expressed by the user. In a non-limiting example, a user may add tag information consisting of sleep data, exercise data, mood ratings, questionnaires, custom tags/notes, blood glucose, body weight, or any other data that is useful for assisting the user in achieving their goals.

Additionally, the user may link an established account maintained on the system server with 3^{rd} party applications and services to automate the collection and display of other types of data that may be associated with the collected HRV reading data and scores, including an established morning readiness score and composite scores reflecting HRV data and ancillary data collected from one or more users.

In an embodiment, the signal quality of the sensor is analyzed in conjunction with the full data captured by the sensor during the HRV reading action. The system server is operative to create a novel, customized signal quality rating. This signal quality rating may be provided to inform and educate the user on the validity and quality of the rating when received and reviewed by the user.

In an embodiment, the signal quality of the HRV measurement apparatus is currently analyzed initially and again with the full collected data from an HRV reading. Currently, a proprietary signal quality rating is provided to the user to educate them on the validity and quality of the reading. This signal quality rating is based on internal research determining the degree of confidence in a result given a certain frequency, total amount, and magnitude of signal artifacts from all sources, as compared to the total duration of the reading and the detected patterns present within the reading. The signal quality score is based on published research standards that have been previously created by physiologists and/or research teams, historical population data that has been collected over time, and patterns in prior data received within the same data collection reading or session.

In an embodiment, customized scoring may be generated from the analysis of the received signal data upon determination that the received HRV reading data is in a form that is ready for analysis by the receiving device, where the receiving device may consist of a system server, a smartphone with or without an internet connection, and/or fitness equipment having an internet connection or having an embedded analysis software module. This may occur when the received signal data is free of artifacts and signal corruption. While there are many potential sources of signal corruption, the net effect, regardless of corruption source or sensor type, can be classified as one of two fundamental types. Either:

The beat detection algorithm missed one or more beats that actually occurred, or
The beat detection algorithm detected one or more false beats that did not actually occur.

Type 1 is sometimes referred to as a false negative, type 2 as a false positive. As will be discussed below, these two types of artifacts have their own distinct waveform patterns and properties, such that the corrupted signal can be analyzed, and often times the impact of corruption can be mitigated or eliminated entirely. It should be noted that ectopic beats can exhibit properties of both false positives and false negatives.

When artifacts that may detract from or compromise signal quality are detected, there are numerous ways to handle the artifacts to clean or correct them programmatically. The HRV system uses a proprietary blend of algorithms for different scenarios to analyze and clean the signal from the data collection sensor or device, as needed. Attempts to clean the signal and improve the data collection effort may include feedback to the user in certain circumstances. In a non-limiting example, the system could suggest to the user data collection times to take readings to improve data collection (as with the Morning Readiness score), suggested postures associated with certain artifacts to remove the generation of these artifacts, etc. Moreover, signal clean up (e.g., filtering techniques) may be applied differentially based on detection of known issues such as incorrect posture.

All artifact detection algorithms presented herein function with the same general logic:
1) Quantify what it means to be “normal”
2) Define a threshold difference from “normal” at which the value is to be declared artifactual.

The variation in sophistication between artifact detection algorithms is hidden within the definition of “normal”. For example, the naive approach to artifact detection, known as “simple thresholding” follows the same overall approach as more effective algorithms, but fails to quantify normalcy in an intelligent way. As suggested by its name, simple thresholding involves selecting some high heart rate value, such as 240 bpm, and some low value, perhaps 20 bpm, and marking any observed value outside of these ranges as artifactual. As one might expect, this system suffers from extremely high false negatives (declares an interval not an artifact when it actually was). One may narrow the values closer to an estimated average, but this only serves to trade false negatives for false positives. The simple thresholding described so far is a static variant, in which the threshold values are not modified per signal. A dynamic variant would be one in which the mean of the signal is calculated, and the thresholds set as mean+c1 and mean−c2 for some constants c1, c2. In fact, this system suffers the same weaknesses as the static scheme.

In an embodiment, a slightly better approach to the dynamic simple thresholding described above would be to replace the mean with the median, and the c1, c2 values with c1*std, c2*std, where std is the signal's standard deviation. Adding this flexibility to the algorithm helps account for the large difference in non-artifactual variance observed across individuals. Still, this system suffers from critical flaws. Most notably the standard deviation of a signal is quite sensitive to artifacts itself. Therefore, if the signal contains a large number of artifacts, or a few artifacts of large magnitude, this system will allow the less deviant, but still artifactual intervals through.

Two innovations in the “normalize and threshold” schema produce significant improvements in detection accuracy. The first innovation is to analyze not the IBI intervals themselves, but rather the differences between subsequent intervals. This strategy minimizes the negative impact of valid local variations in heart rate, while retaining the ability to capture artifact generated spikes or impulses. The second innovation is quantile-based threshold determination. The Berntson algorithm is an industry standard which utilizes both IBI difference analysis and quantile thresholding to good effect. This algorithm assumes a normal distribution of beat differences in order to calculate the Maximum Expected Difference (MED) for veridical beats, and well as the Minimum Artifactual Difference (MAD).

IQR=Q3−Q1

QD=IQR/2=SD/1.48 (assuming gaussian)

MED=3.32*QD

MAD=(Median−2.9*QD)/3

Where QD is the quartile deviation of the IBIs. The artifact cutoff threshold is then taken as a mean of the two values, which given normally distributed IBI differences will cover at least 97.5% of artifact-related differences, though in practice the number is often higher. Additionally, we have made two modifications to the Berntson algorithm in response to empirical testing on data from our user base. The first modification is regarding the logic which marks artifactual beats given threshold-exceeding IBI differences. In principle, the Berntson algorithm marks pairs of IBI's, not individual IBI's, which can be seen as one cost-of-difference based method. The second modification is a set of heuristics for identifying contiguous runs of artifactual beats. While uncommon in most test data sets, the reality of consumer heart beat data is that it frequently contains sequences of spurious beats due to motion artifacts. For any artifact detection method based on IBI difference this presents a problem, since for a run of 3 or more artifactual IBIs, the outermost ones may have threshold exceeding differences, while the inner ones may not.

Another artifact detection technique with traction in the literature is based on impulse response detection. The strategy is to calculate a series of deviations from the median in order to detect unusually large impulses, then normalize each of these differences with another median derived value specific to each RR series.

A windowed version of this algorithm enhances accuracy by cutting the target series into overlapping windows and calculating the median and normalization factor for each window separately. It also sets the overlap factor such that each value (except first few) are tested at least twice. The series of normalized differences is calculated as follows:

Xj(h)=(Wj(h)−Wmj)/med{|Wj(h)−Wmj|}

Where Xj(h) is the normalized difference from the median of the hth element in the jth window. Note that the median in the denominator is calculated once for the entire window.

In an embodiment, a pattern-based windowed impulse response (PWIR) algorithm was tested for which good performance on non-pathological R-R datasets was reported. PWIR functions similarly to WIR except that the sign of differences from median is preserved in order to be able to match specific artifact shapes/patterns. Patterns fall into three categories that determine the appropriate corrective action to perform on an artifactual RR. Possible corrective actions include interpolation and recovery of split intervals via addition. The benefit of this method is that it tests not only the magnitude of an impulse, but the shape formed by every four consecutive samples. This allows for stricter threshold values without major increase in false positives.

### Categories:

1. Missed beat
   a. Shows as positive single spike in RR series.
   b. i.e. False Negative
2. Spurious beat
   a. Negative spike with 1+ points (generally 2 points)
   b. i.e. False positive
3. Ectopic beat
   a. Single point negative spike followed by single point positive spike.
   b. Ectopic beat prevents normal beat from occurring and then subsequent beat come on original schedule.

Note that PWIR seems not to be designed for data contaminated with significant motion artifacts as the case of evenly split spurious beats is unhandled. While the authors clearly state that it is intended for certain usage, that should not be much consolation to developers whose applications consume data from disparate and unpredictable sources.

The final algorithm tested is one based upon the Integral pulse frequency modulation (IPFM) model of heart rate variability. The IPFM model, also called the “integrate and fire” model, describes the beating of the heart in terms of sympathetic and parasympathetic inputs to the sinoatrial (SA) node via a modulating function of time: m(t). The model states that the SA node accumulates these inputs until reaching a critical threshold, at which point a heartbeat is triggered and the integrator resets.

Ψ=∫ₜ_{ᵢ}^{t}^{ᵢ₊₁}m(t)dt

Where Psi is the critical threshold. According to IPFM, the heart beat timing differences are band-limited by the modulating signal, which is itself band-limited. The present algorithm exploits this limitation by estimating the derivative of the instantaneous heart rate signal, and determining where it exceeds a threshold derived from the IPFM model.

The premature contractions known as ectopic beats are even common in healthy individuals. These contractions can be either ventricular or atrial in origin, and are quite distinguishable from normal beats on an ecg. The ventricular ectopic beats can be further classified into ones in which the normal heart beat following the artifactual beat occurs “on schedule”, and supraventricular ectopic beats in which the normal heart beats are effectively “reset” by the artifactual beat. Known patterns such as these can be exploited by artifact detection algorithms. Additional physiological sources of artifacts include atrial fibrillations, ventricular fibrillations, and muscle contractions.

Other artifact sources can be classified as “technical” in that they arise from shortcomings or improper application of the sensor technology. Among these, “movement artifacts” are the most problematic. Because both electrical (ecg) and optical (ppg) sensing modalities work with minute signals, any variations in the distance from the sensor to the surface of the user's skin can induce variations in the signal which are indistinguishable from heart beats, or otherwise reduce the signal to noise ratio such that the true heart period information is not recoverable. Poorly fastened electrodes can exacerbate this problem. Various algorithms exist which attempt to filter out movement artifacts via correlation with a concomitant accelerometer signal, though these algorithms vary between sensor platforms and are not within the scope of this paper. Additionally, there is no accepted standard for QRS complex detection, thus introducing the risk of poorly designed algorithms for an unvetted sensor platform. Beyond the detection of peaks, many sensor platforms attempt to clean up the signal by applying smoothing filters to the Mb. While this practice can improve heart rate measurement stability, it can highly distort HRV metrics.

### Artifact Correction

In order to further evaluate the efficacy of various artifact detection algorithms, the system continues with the heart rate data analysis towards the ultimate end of extracting a useful time, frequency, or nonlinear parameter. Before extracting the parameter of interest though, artifact annotations supplied by the previously discussed algorithms are utilized. The naive approach to artifact correction is to delete them. While this strategy is acceptable for the evaluation of certain time-domain parameters, in particular SDNN and SDANN, it induces significant error in other cases. Frequency domain parameters are particularly sensitive to interruptions in the signal.

Generally speaking, artifactual R-Rs are interpolated rather than deleted. Performant interpolation methods described in the literature include linear interpolation, non-linear predictive interpolation, and cubic spline interpolation. While different effects have been reported for varying interpolation methods depending upon the parameter of interest and data source, the difference between non-deletion interpolation methods may not be significant at artifact rates below 5%. In the system signal processing pipeline, multiple interpolation methods are implemented, with the specific choice determine by which HRV parameter is being calculated. To fairly compare the deleterious impact artifacts on an HRV metric, a consistent method is used, such as, in a non-limiting example, cubic spline interpolation, for the annotations produced by all three detection algorithms under review.

In addition to manually designing algorithms to reduce signal noise and improve HRV scoring from lower quality data, a large amount of historical HRV user data may be leveraged to provide more accurate HRV scores using lower quality data or less data. This additional data analysis allows for HRV scoring to be completed in a shorter time span or completed with data of a quality than is otherwise not currently possible or optimal. In a non-limiting example, a user can provide less HRV data or provide HRV data of lower quality and receive a valid HRV score, perhaps tempered with a score quality or confidence rating.

The HRV system may use machine learning to associate the presently input HRV data with a particular class or category based on a model trained with previously recorded HRV data and scores. A user's input of lower quality HRV data (whether due to sensor type use or amount of HRV data collected) may be insufficient to assign an HRV score using normal processing via a static algorithm. However, the lower quality input HRV data may be input to a machine learning algorithm trained using the pre-existing HRV data currently collected and stored in the HRV system's database. The HRV system may optionally utilize contextual data or a composite of signals to boost the quality of the collected HRV data and thus provide an HRV score of higher confidence in terms of accuracy and quality. In a non-limiting example, the machine learning algorithm may be trained using historical HRV data from a validated sensor. This permits the software to provide an HRV score even though the data collected typically would be insufficient. A reported quality score may indicate the technique used or the confidence of the score reported.

It is worth reviewing the causal sources of artifacts in order to best anticipate and handle them. Artifact sources can be thought of as being either physiological or technical in origin. Physiological artifacts occur when an electrical impulse is generated by some mechanism other than the depolarization of the heart's sinoatrial node.

In a non-limiting example, a novel, customized HRV score may be calculated from the analysis of the received HRV reading data. The system may receive the R-R intervals directly from a chest strap heart rate monitor or other sensor device attached to a user. Obvious artifacts within the data, such as readings that are out of bounds, obviously incorrect, or corrupted, are cleaned and/or removed. The raw, unaltered R-R intervals are backed up securely to an electronic database maintained within the system server. This allows for optimization and improvement of algorithms for all current and past calculations, as well as for the export of the raw, unaltered R-R intervals to a different system or storage location if desired by the medical practitioner or user.

In an embodiment, an additional novel and proprietary score, the morning readiness score, may be prepared by the system and transmitted to a user on a daily basis, in the morning and based upon a morning readiness HRV reading performed by the user. The Morning Readiness gauge indicates a user's state of relative balance. In other words, it is comparing the user's HRV values to the recent past and providing a comparison for the user on whether the user's Autonomic Nervous System (ANS) is in a similar state or if it is swinging widely outside of the norm for the user.

In an embodiment, additional scores may be calculated based upon additional physiological data in addition to collected HRV data. Such additional physiological data, such as image data, environmental data, historical health history data, or other biometric data may form the basis for one or more biological health scores that include HRV data as a particular component. The collection of camera image-based, physiological data may be performed utilizing visual light and/or infrared cameras pointed at the face of one or more users. The collected image data may provide insight into biometric data such as heart rate, blood pressure, temperature, oxygen levels, CO2 levels, glucose, ketones, general awareness or alertness, stress, reflex time, resilience, or a combination of any of these data categories. The resultant combined score may include HRV data or may consist of collected biometric data as a corollary to a calculated HRV score.

In an embodiment, the HRV system may provide users with the benefit of score and performance analysis to assist in predicting success with short-term and long-term physical goals and recommendations and suggestions on how to achieve identified user goals.

To achieve such predictions and recommendations from the HRV system users can submit data related to their goals, plans, HRV data and outcomes and utilize the HRV system to identify and/or formulate optimal plans to achieve their desired goals. In a non-limiting example, such a recommendation may take the form of a general training plan or a training plan customized for the individual. Community members may vote on these plans to surface the best plans, which could be promoted to users, e.g., based on HRV data similarity to those that have completed the plans.

In an embodiment, the HRV may also provide Artificial Intelligence (AI) enhanced and implemented performance predictions and plan suggestions. These predictions and plan suggestions may take the form of a virtual coach, but specifically incorporating HRV data as an input. These AI suggested plans or virtual coaches may take the place of user submitted plans. To implement AI suggested plans, HRV system may develop machine learning algorithms that take user profile data, including HRV data, and use it to predict the type or level of exercise to suggest to the user to achieve a specific goal. Similarly, this profile data, including HRV data, may be used to predict performance during an activity, such as running or biking. Additional types of program suggestions could be implemented outside of the health and fitness domain while still making use of HRV data. The additional program suggestions may realize the benefit of the scoring provided by the HRV system to create a service for users.

The HRV system may leverage its ability to accurately analyze HRV data as a service to others. In a non-limiting example, the HRV system may offer a scoring service by which the HRV system receives HRV data collected by a third-party application, analyze the third-party collected HRV data as a service to a user or third-party entity, and output the analysis to the third-party app for use by the third-party app. This service offering may include receiving and ingesting the collected raw HRV data as a cloud service or offering an API to third parties for data ingestion, processing the raw data, and outputting proprietary score(s) to the requesting application. This service may be offered by the HRV system and used to operate on a variety of different input data types and produce a variety of different HRV based scores, system and application modifications, or data displays.

In an embodiment, the HRV system may also provide trend and analysis information based upon HRV data collected and scores derived from the HRV data collected. In a non-limiting example, the Morning Readiness score calculated by the HRV system may provide a daily baseline indication for the user. The Morning Readiness score is trended and charted over time to help the user understand how acute, short-term, medium-term, and long-term choices and events impact the score over time. In another example, the HRV Score and other data and parameters can be charted and analyzed longitudinally, as well as for each individual reading.

The large amount of existing HRV user data may allow the HRV system to provide more specific guidance to users in view of the user's trend data. In a non-limiting example, the HRV system can discover, either utilizing a manual review or an automated machine learning process, that prior users exhibiting a similar trend had a positive or negative outcome by making certain adjustments. These data insights can form the basis of customized feedback for the users given their data trends, desired outcomes and past user experiences. In this non-limiting example, the HRV system may associate a current user's trend data and a stated goal (e.g., mental health, weight loss, etc.) with other users having similar trend data, known modifications (e.g., increased exercise, decreased sleep, etc.), and the same or similar stated goal. Having this information, the HRV system software can suggest changes that have been helpful for past members and provide cautionary information about modifications or continuations of the same behavior that have been historically harmful or negative for members in the past.

Additionally, the HRV data may indicate inflammation in the body and may be analyzed to create an inflammation score for tracking adverse conditions, also forming a portion of the tracked health data. Also, in addition to analyzing a user's own data, the user has the option to link their data to a team, where a coach, wellness practitioner, or medical practitioner may view the data.

In addition to requests to the HRV system for analysis of their own data, users have the option to link their own collected data to a team or group, where a coach or healthcare practitioner can view the data. The coach or healthcare practitioner in turn may have access to team level and individual team member level HRV based feedback, such as proprietary scores, customized modifications to training plans, etc. This allows the users, e.g., coaches, trainers, healthcare professionals, to access customized guidance for clients, patients, etc., e.g., at the team or organization level, subgroups within the team or organization, or individual team or organization members. This permits group leaders to have access to HRV data of the team or group and associated HRV-based guidance with increasing specificity. In a non-limiting example, a CrossFit gym may obtain an HRV-based suggested modification (e.g., color coded Green/Yellow/Red indication) to the workout of the day (WOD) for individual users or groups of users. This would allow a personal trainer to understand which users are capable of strenuous, moderate, or light exercise that day and have access to suggested modifications to the workouts. These modifications may be selected based on global data (e.g., other users having similar HRV readings or trends) or more specific data, e.g., coach or healthcare professional modifications matched to HRV recommendation categories.

In this example, a matrix display may be provided for dynamically organizing team or group members per HRV system-based suggestions or modifications. A variety of user interfaces and functionalities may be provided in connection with a team-based view. To support this view the HRV system may provide a capability to sort team members by HRV-based workout intensity recommendation. In an exemplary embodiment, a matrix may be displayed organizing the team or group members into columns and rows, such as one user per row, with a color coded (or otherwise indicated) HRV based modification, along with an HRV score in associated columns. These HRV system-based modifications may be paired with predetermined, customized guidance per user, such as that input by a coach, health practitioner, etc. As above, the matrix can be re-organized to dynamically group users via various modalities. The HRV system may prepare the matrix listing users per sub-group (e.g., offense and defensive positions), based on HRV scores (or ranges), based on modifications, or based on any grouping that provides useful information to the user.

In an embodiment, the HRV system may be implement utilizing a finger sensor based on LEDs that collect PPG data. The finger sensor uses three LEDs (infrared, red and green). The LEDs are paired with sensors (detectors) on opposing sides. The LEDs cycle to attempt to obtain a strong reading, which assists in handling user differences (skin tone, cardiac patterns, etc.). The LEDs take readings at 500 MHz. The current sensor can measure other data, such as pulse oximetry data, in addition to HRV data. However, the HRV system data collection readings may be performed utilizing other sensor devices including gaming input devices, AR/VR gloves, or other physical sensors. The HRV system may accept HRV data collected by any available hardware device that provides sufficient signal quality to collect the HRV data at acceptable sample rates.

In an embodiment, instead of utilizing an external sensor the HRV system may collect HRV data using an integrated sensor such as a wearable device that collects HRV data natively. Examples of such integrated sensors may include devices such as, in a non-limiting example, an Apple Watch, or a smartphone or other computer-based camera that facilitates image-based HRV data collection, coupled with other data collection (e.g., blood pressure, pupil dilation, device data such accelerometer, etc.). Use of existing sensors of the user's common hardware (e.g., smartphone, smartwatch, laptop, etc.) may extend the ability to collect HRV data more conveniently and provide more users and data. Of these sensors, cameras and finger-based physiology detection sensors are among the few currently viable options. These have been used by others for obtaining HRV data. These sensors may be improved by reducing finger movement via reduced reading times or finger stabilization techniques utilizing a magnetic accessor that attaches to the finger to stabilize it, etc.

In an embodiment, in addition to suggested modifications to work out plans or health or wellness treatments, such data can be used to validate treatments, for display or feedback by gamers or those watching a live streaming event. The data may be utilized in an office to determine when employees should take breaks, to guide meditation or breathing practices using live, real-time feedback, to create, modify or evaluate the efficacy of corporate wellness programs, and in stress level monitoring. The HRV data may also be used in content recommendation systems, to enhance sports broadcasting and news broadcasting, or to modify the behavior of systems or devices, such as the behavior of automated vehicles, self-service kiosks, gaming systems, advertisement or content selection systems, smart home devices, office furniture, etc. In a non-limiting example, the user's detected HRV data or a score using the collected HRV or other data may be used to influence advertisement selection (alone or in combination with other contextual data, e.g., GPS location of the user's device) or to influence music selection systems to change music based on a user's determined mood or goal for the day (and the current progress towards that goal). The collected HRV data could in turn be fed into other device applications, e.g., virtual assistants or smart home devices to adjust their recommendations, tone, etc., or to adjust office furniture, room temperature, ergonomics, and sleep environment. As noted above, such scores or suggested modifications may be provided as a service to various third-party applications and devices.

In an embodiment, the HRV system may collect and accumulate HRV data, HRV scores, camera sensor-based image data, and/or other biometric data for a user. This data may be acquired from various sensors and sources of data and stored in an electronic storage apparatus. The HRV system may then look to the user to define a goal with regard to improving one or more HRV and/or combined HRV and composite data scores that the user wishes to achieve and provide a suggested activity to achieve that goal. The activity suggested for the user may be one part of a predetermined plan based at least in part on a model trained using HRV data, HRV scores, camera sensor-based image data, and/or other biometric data.

In an embodiment, the HRV system may obtain biometric data for a plurality of users, including HRV data collected from a sensor, and utilize the biometric data to train a model utilizing machine learning algorithms. The machine learning algorithms may classify the biometric data into one or more predetermined classes. This collected data may then be analyzed by the HRV system to predict an HRV score, or a composite score, during or after completion of a pre-established activity. The HRV and composite data may be normalized to reflect population trends and help a user understand their particular scores as compared against population averages and norms.

In an embodiment, the HRV system contains a method for biometric monitoring and scoring in which the HRV system is actively collecting biometric data from a plurality of users. The collected data measures physiological data and environmental data associated with said plurality of users over time and stores all collected data in an electronic storage system. The HRV system is then active to analyze all collected data to create a composite score that is based at least on heart rate activity data, biometric data, and environmental data. The composite score may be compared against historical composite scores to determine activity modifications that will impact the behavior of any of a plurality of users prior to collecting additional biometric data. The HRV system may present these activity modifications to the plurality of users on a display device such as a wearable device, smartphone, or other mobile device in combination with recommended actions to accomplish said activity modifications.

In an embodiment, the HRV system may utilize one or more signal cleaning algorithms to detect artifacts in the collected data and improve the collected data by removing any detected artifacts that impair the signal quality of the biometric and/or physiological data. Additionally, the signal cleaning of the collected data may be performed during a data collection action and the cleaned collected data is stored in an electronic format prior to analysis of said cleaned collected data. The HRV system may track the composite score over a pre-configured time span. The composite score may consist of at least HRV data, biometric data, physiological data, and environmental data associated with a single user or a group of users. The HRV system may receive from a user or medical practitioner a threshold composite score or composite score range that is preferred for the user to maintain. The HRV system may transmit to a user recommended actions comprising events, interventions, and/or planned steps in accordance with maintaining said user's particular composite score.

In an embodiment, the HRV system may utilize any of a finger sensor, an LED sensor, a chest-strap electrocardiogram sensor, a camera, or sensors contained within or attached to a mobile device associated with a user. Once calculated, the composite score may be presented to a user as a numeric value and a gauge graphic to permit the user to visually understand changes in the composite score over time. Additionally, the composite score, recommendations, and guidance may be provided as a report, as part of an ongoing data display, or in real-time as live biofeedback to a user during an activity.

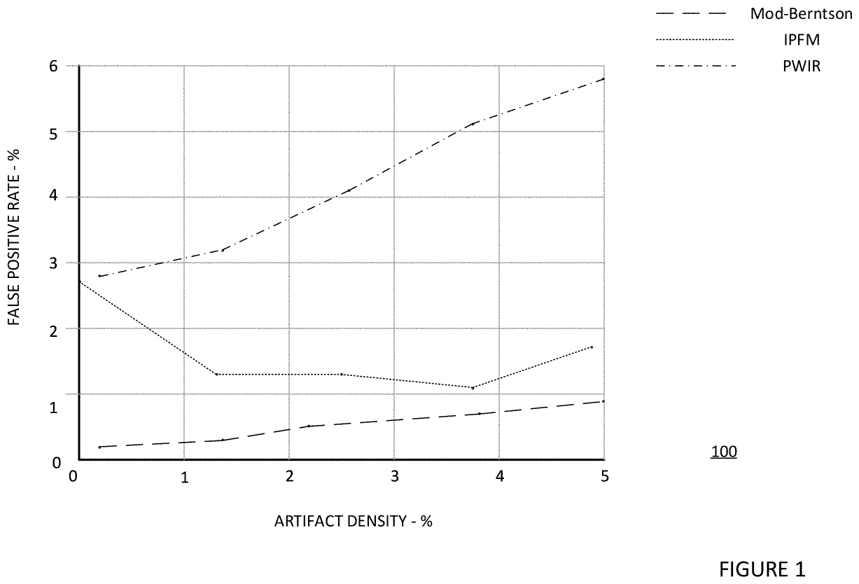

Turning now to FIG. 1, this figure presents a view of artifact detection accuracy in terms of the detection of false positive artifact detection consistent with certain embodiments of the present disclosure. In an exemplary embodiment, at 100 the mean false positive rate is the ratio of falsely annotated artifacts to the total number of veridical intervals such as, in a non-limiting example, negative artifacts. The false positive artifact detection is performed utilizing the Mod-Berntson, IPFM and PWIR processes. This figure displays the relative accuracy of each method when compared directly. The exceptionally high False Positive Rate (FPR) of PWIR can be mitigated by increasing its threshold parameter, but not without inducting a reduction in false positives on missed beats which hurts the HRV estimation mean error more than the false positives do.

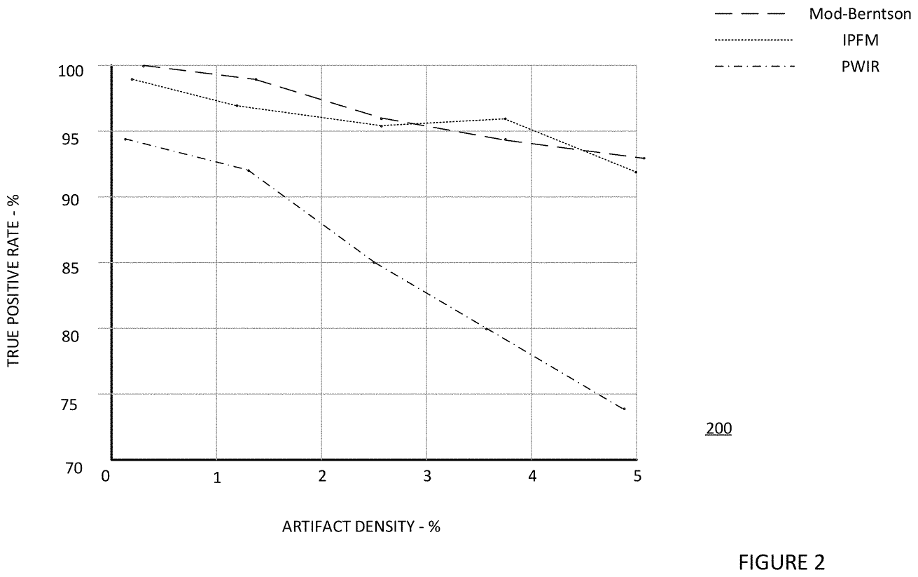

Turning now to FIG. 2, this figure presents a view of artifact detection accuracy in terms of the detection of true positive artifact detection consistent with certain embodiments of the present disclosure. In an exemplary embodiment, the system presents a mean true positive rate is the ratio correctly identified artifacts to the total number of artifacts 200. While not shown here, the performance of IPFM when evaluated exclusively on spurious beat-type artifact is actually exceptional. Unfortunately, the instantaneous-derivative metric used by IPFM as a threshold metric is not nearly as sensitive to missed beats, which hurt its overall performance significantly. It was also found that the median FNR value on all artifact conditions for the Modified Berntson algorithm was 0%.

The IPFM and WPIR algorithms were applied using threshold parameters recommended by Osman et al. Further analysis might include a complete parameter search against the present test data. It is worth noting that the modified Berntson algorithm robust across data conditions given its standard parameterization.

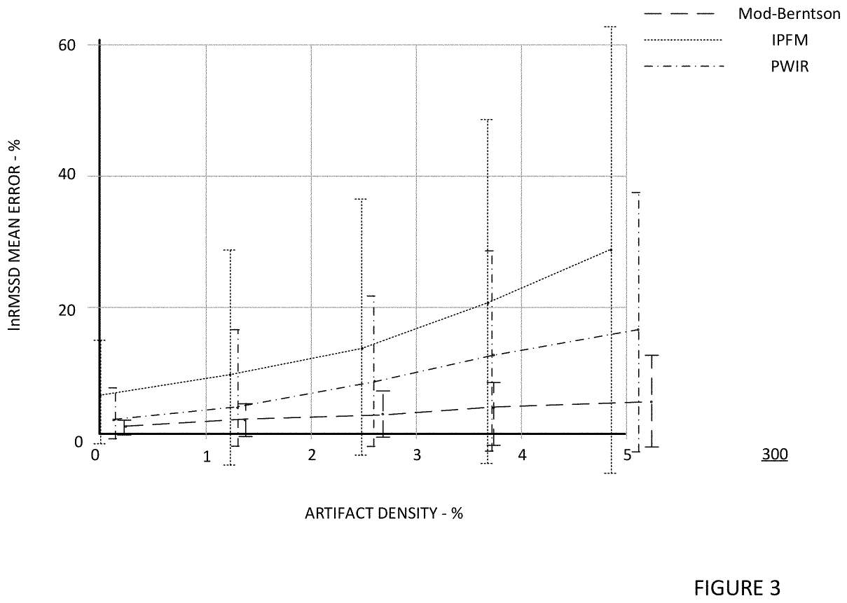

Turning now to FIG. 3, this figure presents a view of artifact impact on the system consistent with certain embodiments of the present disclosure. In an exemplary embodiment, at 300 when calculating the impact of artifact on the system the best performance for IPFM was found with threshold set to 4.5, and for PWIR set to 2.5. These thresholds are used in all included plots. The optimal threshold can differ significantly with data source, therefore performance of these algorithms on a data source for which there is no prior knowledge may be worse. Alternatively, no parameter optimization was performed for the modified Berntson algorithm. This property of being functionally parameterless is of considerable value when no opportunities for pre-emptive tuning or source analysis are available.

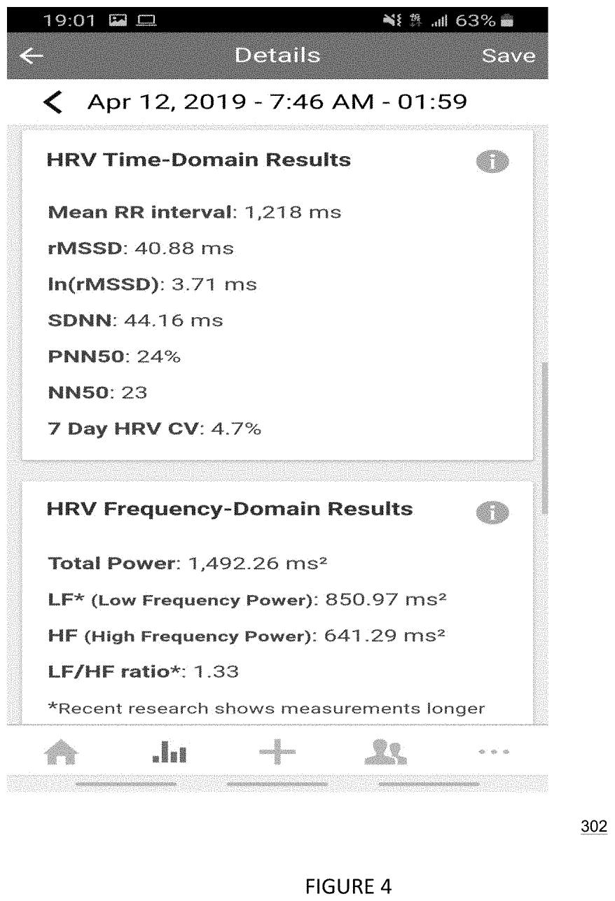

Turning now to FIG. 4, this figure presents a view of the display of HRV statistics for a user post reading consistent with certain embodiments of the present disclosure. In an exemplary embodiment, at 302 the display presents a display of the time-domain and frequency-domain results for the HRV reading taken by a user or by a medical practitioner on behalf of the user. The time-domain statistics may include, but are not limited to, the mean RR interval, rMSSD, ln(rMSSD), SDNN, PNN50, NN50 and a 7-day HRV Calculated Value (CV). The frequency domain statistics may include, but are not limited to, the total power consumed, the Low Frequency (LF) power consumed, the High Frequency (HF) power consumed, and the ration of LF to HF power.

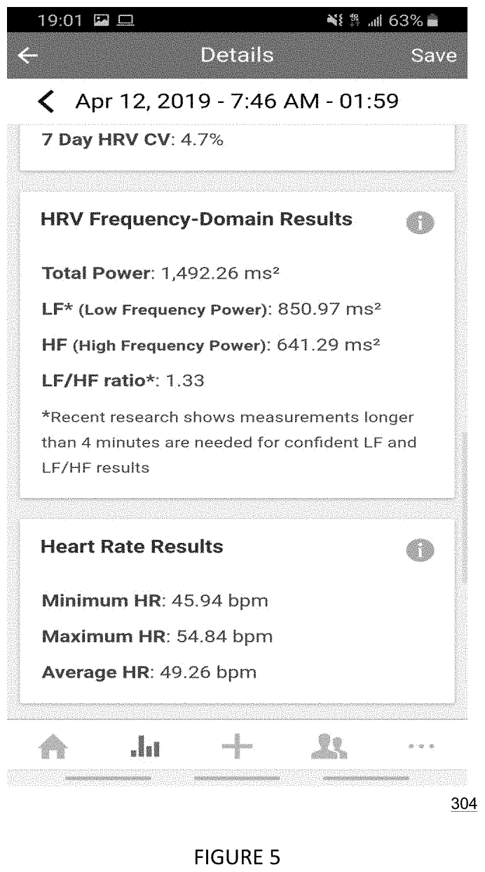

Turning now to FIG. 5, this figure presents a view of the display of the continuation of HRV statistics for a user post reading consistent with certain embodiments of the present disclosure. In this embodiment, at 304 the display presents the display of frequency domain results for the HRV reading as previously described and continues with a display of the user's heart rate results for the reading. The heart rate results statistics presented include but are not limited to, the minimum heart rate, maximum heart rate, and average heart rate captured during the data reading action.

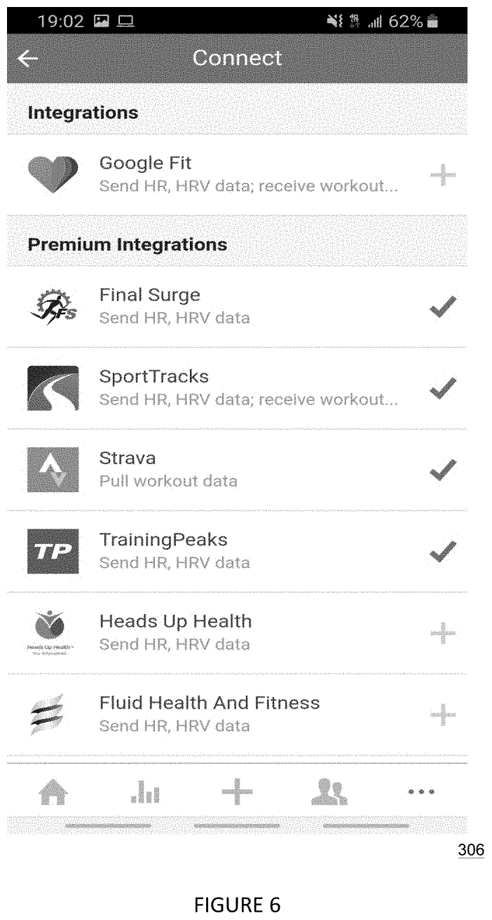

Turning now to FIG. 6, this figure presents a view of the display of the data integration connections for the user device consistent with certain embodiments of the present disclosure. In this embodiment, at 306 the user may specify one or more exterior monitoring devices, such as, in a non-limiting example, a Fitbit or Google Fit device, with which the HRV monitoring device may connect. The exterior device may integrate with the HRV monitoring system to receive, transmit, and exchange heart rate, heart rate variability, exercise regimen, nutrition data, and any other data that assists in monitoring and managing the health of the user. In a non-limiting example, the user may configure the HRV monitoring system to transmit data to any selected exterior monitoring device, or to an exterior monitoring device maintained by a medical practitioner of the user's choice.

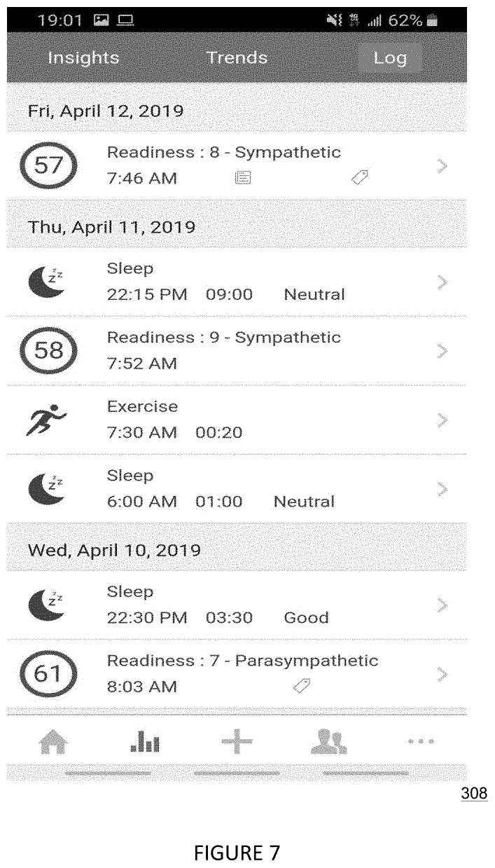

Turning now to FIG. 7, this figure presents a view of the display of the historical log for a user consistent with certain embodiments of the present disclosure. In this embodiment, at 308 the historical log presents data to the user to permit a visual tracking of the morning readiness score, sleep statistics, exercise statistics, and other health information. The data presented may be configured by the user to provide that information that is most useful to the user in monitoring trends for these health-related statistics over time.

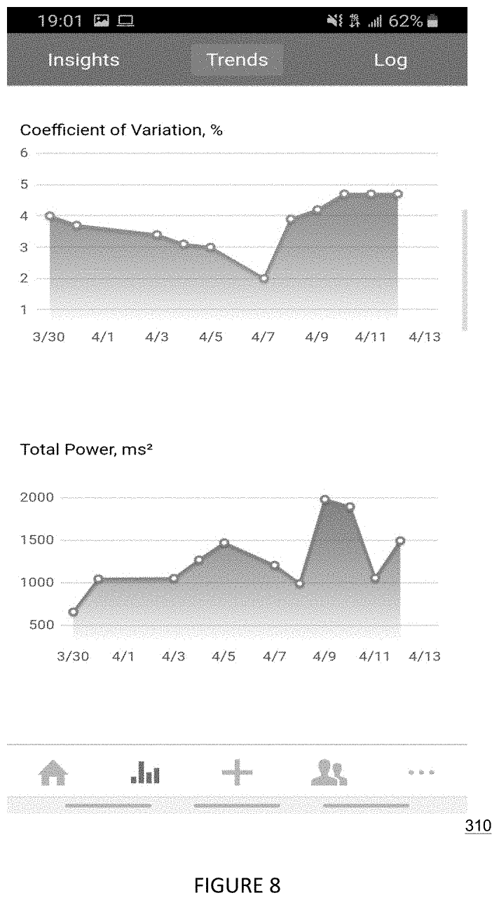

Turning now to FIG. 8, this figure presents a view of the historical trends for HRV statistics for a user consistent with certain embodiments of the present disclosure. In this embodiment, at 310 the displays presented to the user provide a snapshot of statistical information in chart form, permitting the user to understand changes in their health readings and health related statistics over time. In a non-limiting example, a portion of the display presents a chart of a coefficient of variation for the HRV data readings expressed as a percentage and a second portion of the display presents a chart of the total power readings captured over time. However, this example should not be considered limiting as the user may configure the display to present any other statistical measure captured by the system over time. The selected statistical data may then be charted and presented to the user on this display when requested.

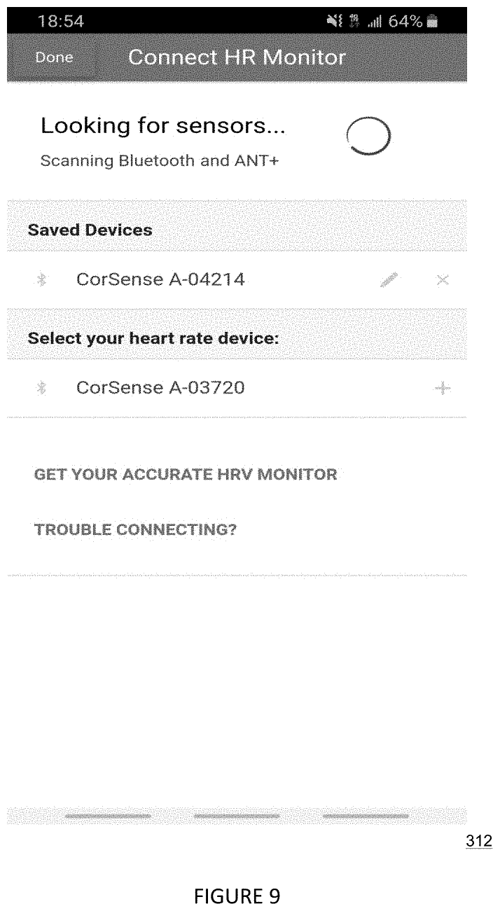

Turning now to FIG. 9, this figure presents a view of the connection capability for the sensors associated with the HRV monitoring system consistent with certain embodiments of the present disclosure. In this embodiment, at 312 the user would open this display to begin the process of selecting the desired Heart Rate (HR) monitoring sensor and performing the connection actions to place the HR monitoring system in readiness to perform a data reading. The HR system presents the user with one or more sensors that have been discovered through an embedded near-field communication protocol, such as, in a non-limiting example, Bluetooth Low-Energy (BLE). The user may then select the sensor they intend to use for the HRV data reading by selecting the sensor name on the display screen. The user is also presented with a troubleshooting capability to resolve issues when a sensor does not connect or indicates errors or issues with connection. Upon an indication of connectivity and readiness, the user may proceed to use the selected HR monitoring sensor to capture the HRV data reading.

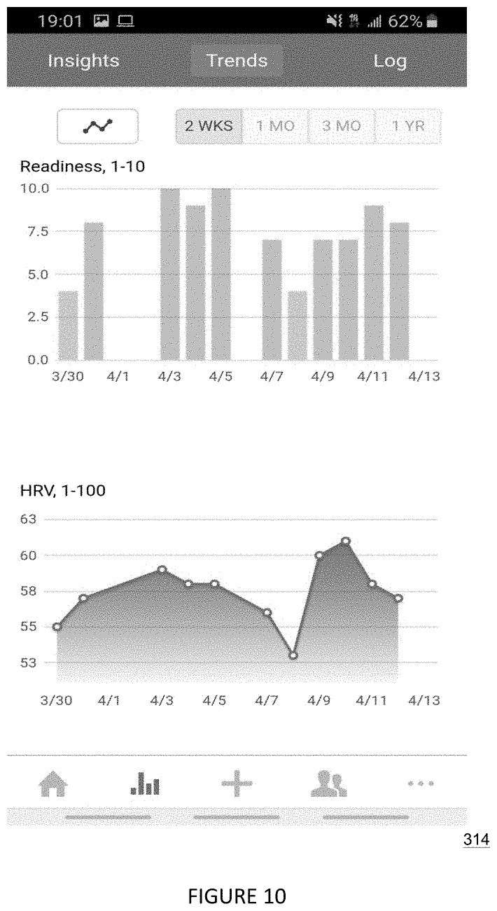

Turning now to FIG. 10, this figure presents a view of the historical trends for HRV statistics for a user related to morning readiness scores and HRV values consistent with certain embodiments of the present disclosure. In this embodiment, at 314 this display presents charts of the morning readiness and HRV data readings over a selected span of time. The user may choose the length of time for the charted information from an icon on the screen indicating the desired time span. The user may also choose to change the chart time span to move from one time-span to another by selecting a different time span icon on the display screen, allowing the user to compare short term and long-term trends.

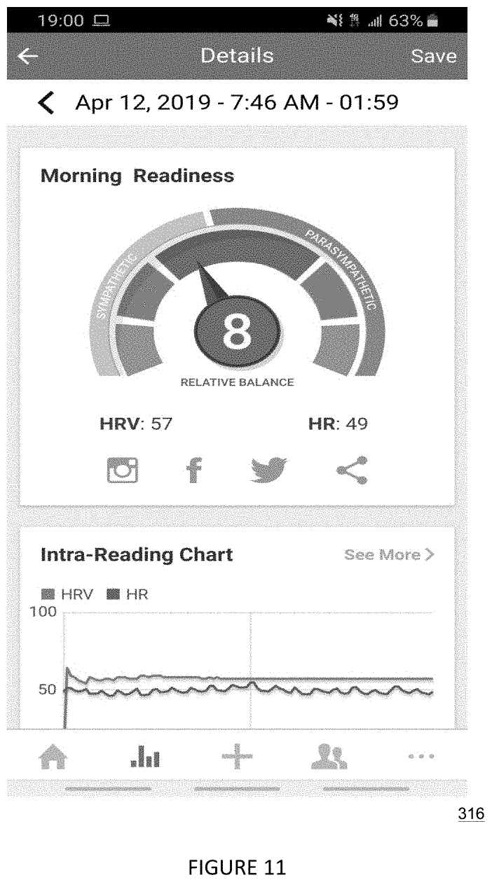

Turning now to FIG. 11, this figure presents a view of the detailed data values for HRV statistics for a user related to morning readiness scores and HRV values consistent with certain embodiments of the present disclosure. In this embodiment, at 316 the user may be presented with an indicator that displays the morning readiness score as a relative measure between sympathetic and parasympathetic conditions to provide a relative balance indicator. This display also provides the user with heart rate and HRV data readings and charts intra-reading values that may be interpolated from the HRV data readings. This detailed information display provides the user with a view into the actual variability in the intra-beat heart rate data.

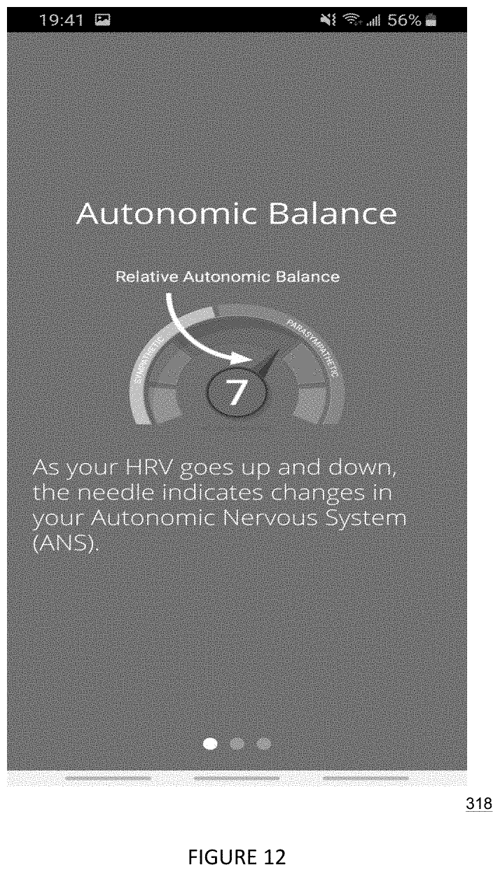

Turning now to FIG. 12, this figure presents a view of the informational data for a user related to morning readiness scores and HRV values expressed as autonomic balance between the sympathetic and parasympathetic systems consistent with certain embodiments of the present disclosure. In this embodiment, the displayed information 318 is educational and informative in nature, providing the user with an understanding of how the morning readiness score indicates a balance between the sympathetic and parasympathetic condition of the user's autonomic nervous system.

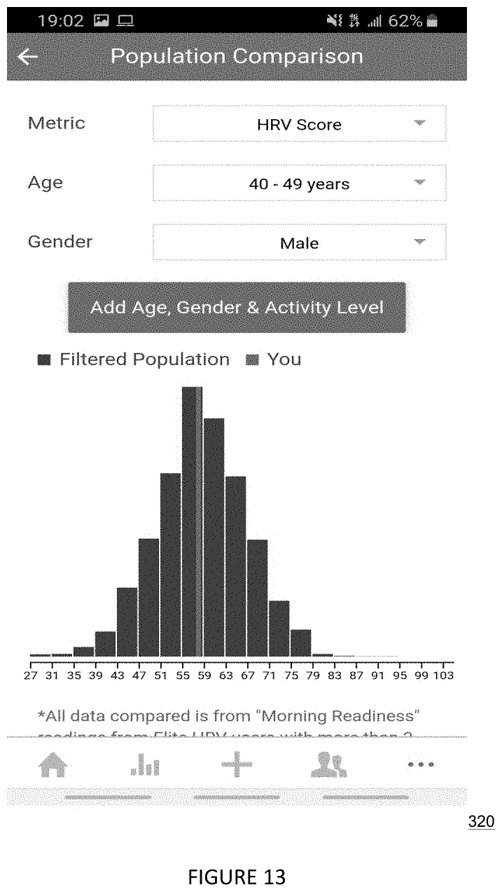

Turning now to FIG. 13, this figure presents a view of the historical trends for HRV statistics for a population related to morning readiness scores and HRV values consistent with certain embodiments of the present disclosure. In this embodiment, the user is presented at 320 with metrics associated with the user and presented as a comparison with a filtered population. The user or a medical practitioner may input metrics associated with a particular HRV score, age and gender to create a comparison between the input metrics and the filtered population as a whole.

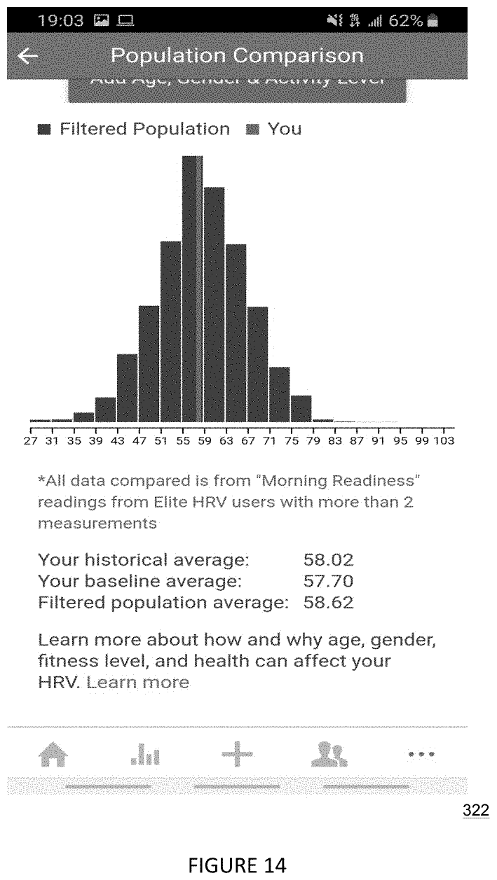

Turning now to FIG. 14, this figure presents a view of the historical trends for HRV statistics for a population related to morning readiness scores and HRV values consistent with certain embodiments of the present disclosure. In this embodiment, shown as a continuation of the comparison view of FIG. 13, the user at 322 is informed that the filtered population is filtered based upon morning readiness readings from all system users having more than 2 measurements stored within the morning readiness score database maintained by the system server. Once again, the information provided to the user on this screen is informational and is intended to educate the user on how and why age, gender, fitness level, and health can affect the user's HRV.

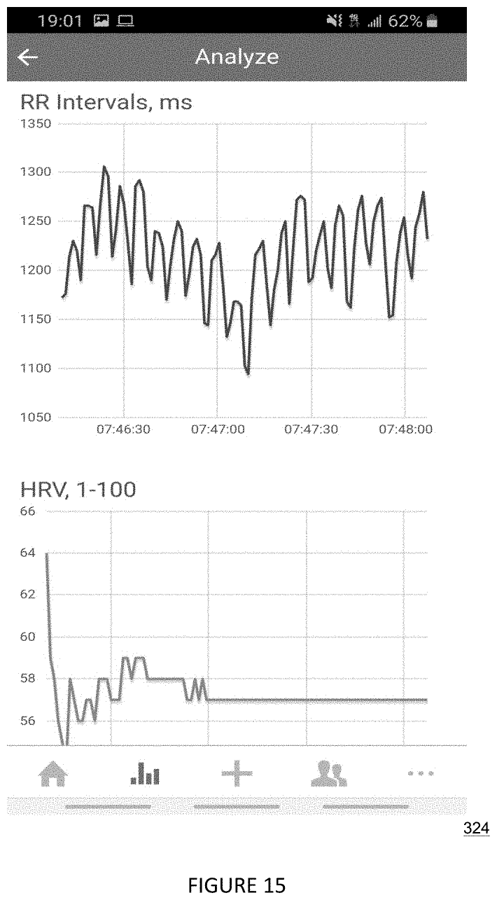

Turning now to FIG. 15, this figure presents a view of the raw data captured for RR intervals and HRV values consistent with certain embodiments of the present disclosure. In this embodiment, the user is presented with the actual data captured by the selected sensor and presented to the user as a chart of values over time 324. The RR interval data is collected over a time span of at least 2 minutes, although the time span may be longer if greater accuracy is desired, and the data is collected continuously over the reading time span. The RR intervals are reported in milliseconds and provide the basis for the determination of HRV, which is charted over the same time span and presented on a normalized scale of 1-100. From this raw data, the user or medical practitioner may have a more optimized view of the user's HRV and the RR intervals that contribute to the HRV values for the time span during the data reading.

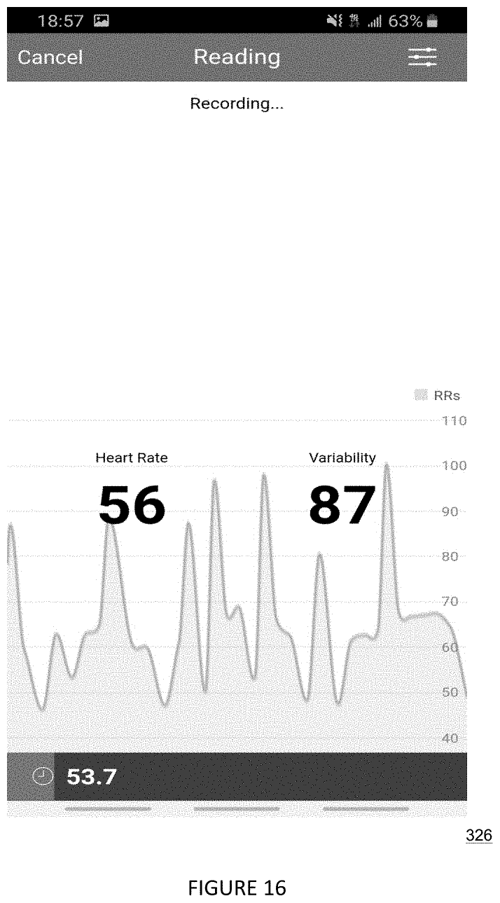

Turning now to FIG. 16, this figure presents a view of the relationship between RR intervals and HRV values consistent with certain embodiments of the present disclosure. In this embodiment, in this view the user or medical practitioner is presented with a comparative chart of a user's heart rate and the associated variability in that heart rate (HRV) for a particular data reading 326. From this view, the user or the medical practitioner may derive a better understanding of the amount of variability the user is experiencing in their monitored heart beats, even though the number of beats and timing may be well within physical norms for the user's age, gender, fitness level, and weight. This particular display may present a user, or the user's medical practitioner, some insight into whether steps should be taken to optimize the user's HRV.

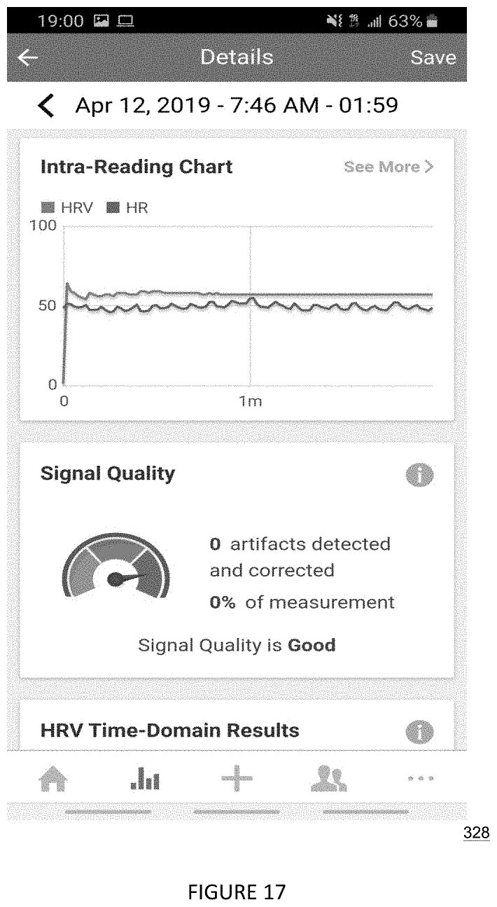

Turning now to FIG. 17, this figure presents a view of the display of HRV statistics and signal quality for a user post reading consistent with certain embodiments of the present disclosure. In an exemplary embodiment, this display presents the user with an operational view of the signal quality for received data from the sensor and the HRV and HR data values collected during a reading period 328. The signal quality presents the user with a view of whether data artifacts are appearing in the recorded data and, if so, how many such artifacts have been detected. If the user is experiencing poor results from a data reading, the user may use this display to determine if signal quality or data artifacts are causing the poor data reading. If either the signal quality or data artifacts are causing an issue with collecting data measurements, the user may take steps to correct the issue.

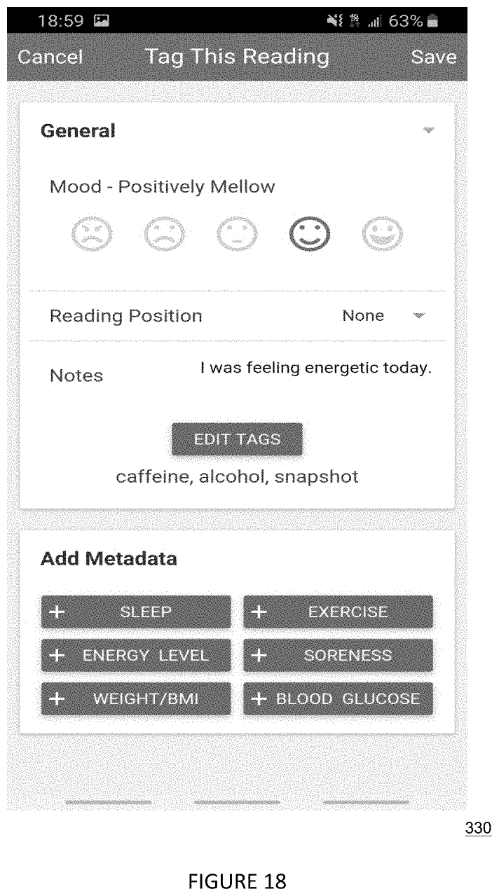

Turning now to FIG. 18, this figure presents a view of the user feedback and tagging display consistent with certain embodiments of the present disclosure. In an exemplary embodiment, the user may utilize this input data view to tag a collected HRV data reading with a mood the user was feeling when the HRV data reading was recorded 330. The user may also use this display to record notes as to physical feelings and edit information associated with the user's interaction with caffeine, alcohol or other chemicals. The user may also add metadata associated with sleep, energy level, exercise, and/or soreness to add to the HRV data reading when it is stored within the HRV data reading data base. This information, although somewhat subjective, may also assist the user in optimizing their heart rate variability over time.

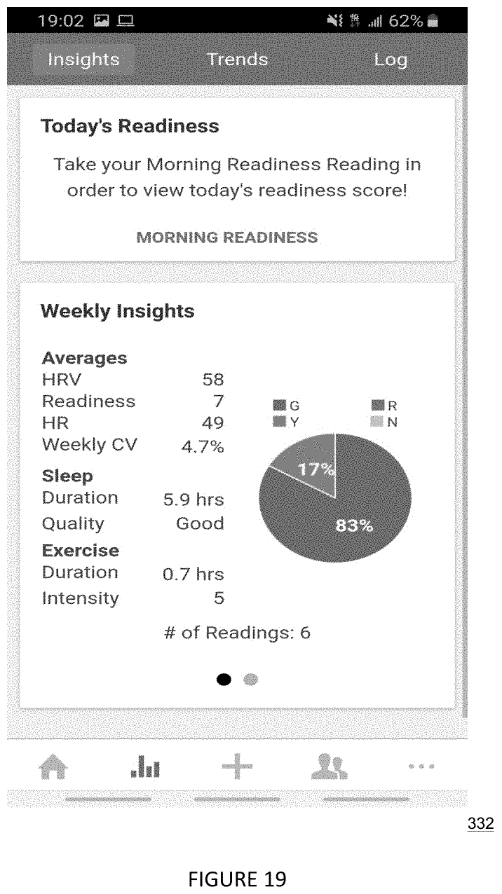

Turning now to FIG. 19, this figure presents a view of the display of HRV statistics and signal quality for a user post reading consistent with certain embodiments of the present disclosure. In an exemplary embodiment, the user may visit this display page to review insights into the user's condition over time 332. Although the display presents the data as a weekly insights display, this time span should not be considered limiting as the user may select different time spans over which to observe the data points presented on the display, once again in an effort to optimize the user's HRV over time.

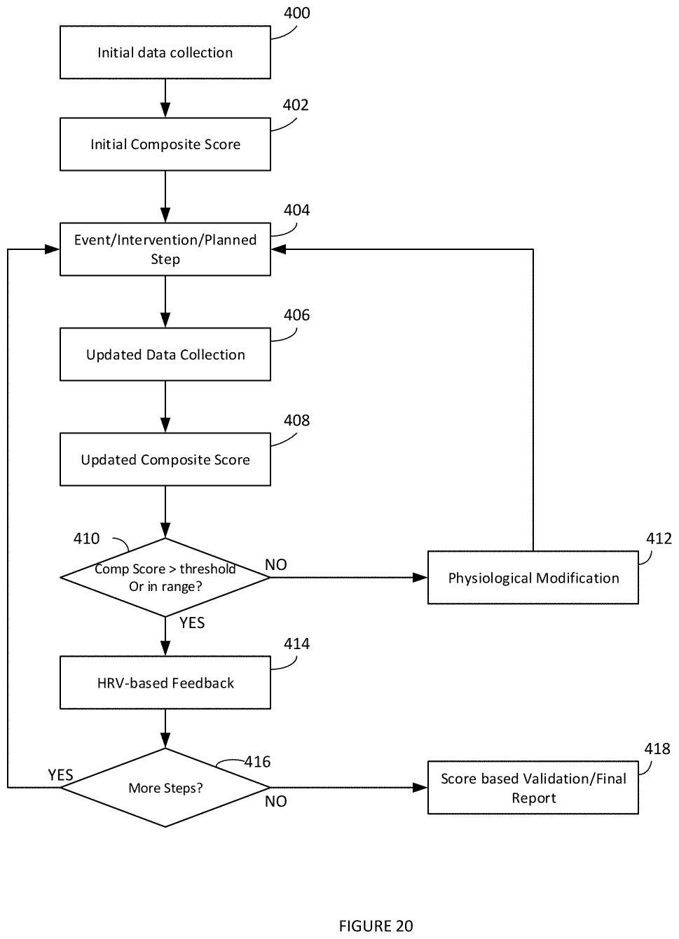

Turning now to FIG. 20, this figure presents a view of the composite reading and data collection process consistent with certain embodiments of the present disclosure. At 400 a user or medical practitioner on behalf of a user initiates a data collection action to collect HRV data, physiological data, biometric data, and environmental data. The initial data collection secures information about the user's heart beat R-R values, environmental data about the area in which a user is located, and other biometric and physiological data such as heart rate, blood pressure, oxygen levels, CO2 levels, glucose, ketones, general awareness or alertness, stress, reflex time, resilience, training or related capacity or capability, and video imagery. The data collected during the HRV reading is stored in the HRV system server electronic data store and combined with historical data and other information to create an initial composite score, comprising HRV data, environmental data, and other biometric data, for the user at 402. At 404 the HRV system may analyze the collected and accumulated historical data to create a planned event, intervention or planned step to assist a user in achieving one or more expressed goals with regard to the composite score and provide this guidance to the user. After the user has performed the communicated planned event or events, intervention or planned steps the user at 406 will perform another data collection action to update the initial data recordings and collect updated data on all sampled values subsequent to the user performance of the planned event, intervention, or planned step. At 408 the HRV system may perform a calculation to update the composite score utilizing the collected data from the most recent data collection effort. At 410 the HRV system analyzes the updated composite score to determine if the latest calculated composite score is above or below a threshold value or within a range that is indicated as desired for the user.

If the updated composite score is not above or below a threshold value or within a range desired for the user, the HRV system updates the planned intervention for the user at 412 by choosing or creating a modification to the previously recommended event, intervention, or planned step and returns this value to the HRV system server. The HRV system server then returns to process step 404 to provide this information to the user. If the updated composite score meets or exceeds the threshold value or is within the desired range for the user the HRV system provides updated feedback to the user on their composite score values and how the user is meeting their goals with regard to the established composite score. At 416 the HRV system queries the user to determine if additional data collection and/or analysis is desired by the user, or by the medical practitioner associated with the user. If additional readings or analysis are desired the HRV system returns to step 404 to provide the updated modifications created for the user by the HRV system and the user performs the remaining steps in the process utilizing the updated modifications in performing those steps. If no further steps are required the HRV system at 418 may produce a score validation for the user and create a final report for the current data collection readings and the user's current state with regard to their expressed goals and/or the composite level goals established for the user by the HRV system.

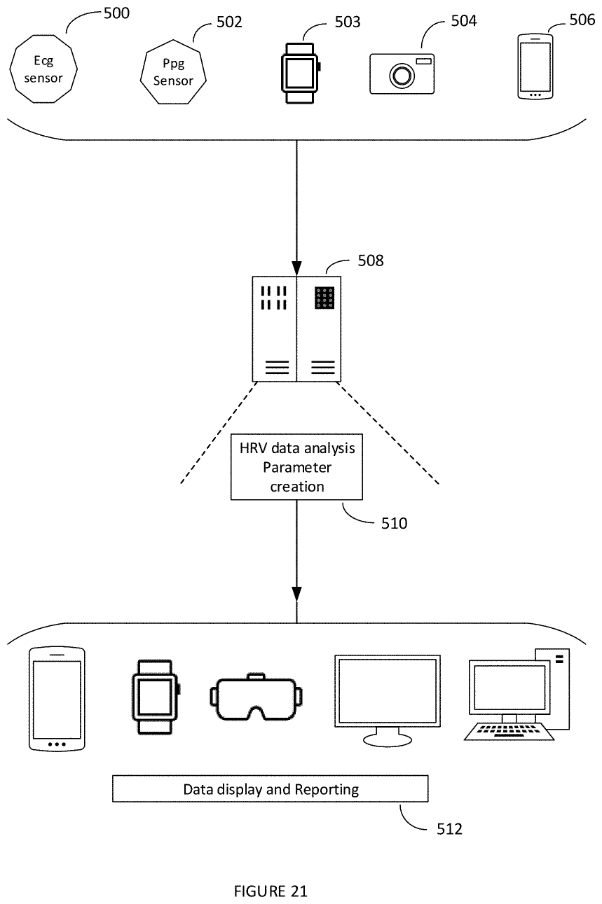

Turning now to FIG. 21, this figure presents a view of the HRV system configuration consistent with certain embodiments of the present disclosure. In this embodiment HRV data may be collected from a user through the use of any sensor or device configured to collected HRV data. These sensors or devices may include capturing the HRV data through attaching an ecg sensor 500 or ppg sensor 502 to the user, or the HRV data may be captured through the use of a camera 504 or using a smartphone 506. The data captured by any sensor or device may be collected and transmitted as a stream of data in real-time, or may be collected and transmitted in a batch at a later time. Regardless of the method of collection or data transmission the collected data is transmitted to the system data processor 508. Within the system data processor 508 a plurality of modules are active at 510 to perform the HRV analysis herein described and creating parameters for review as well as predictions and recommendations for the user. The data, predictions, and recommendations are later transmitted to any display device associated with a user at 512 to display the information for consumption by the user.

While certain illustrative embodiments have been described, it is evident that many alternatives, modifications, permutations and variations will become apparent to those skilled in the art in light of the foregoing description.

1. A method for biometric monitoring and scoring, comprising:
receiving biometric data from a plurality of users, said biometric data collected when users are not physically active;
receiving physiological data comprising image data, historical health history data, and environmental data associated with said plurality of users over time;
combining said biometric and physiological data to create a combined score that comprises heart rate activity data, Heart Rate Variability (HRV) data, and collected biometric data for each of said plurality of users;
comparing said combined score against previously stored combined scores to determine modifications to user actions and activities to permit any of said plurality of users to meet an expressed goal;
presenting HRV-based modifications to user actions and activities in combination with one or more recommended actions related to said modifications to user actions and activities to any of said plurality of users and/or medical service providers associated with any of said plurality of users;
any of said plurality of users implementing said presented HRV-based modifications to user actions and activities to achieve an expressed physiological goal as measured by said combined score.

2. A method according to claim 1, further comprising utilizing one or more signal cleaning algorithms to detect artifacts in the collected data and improve the collected data by removing any detected artifacts that impair the signal quality of the biometric and/or physiological data.

3. A method according to claim 2, where signal cleaning of the collected data is performed during a data collection action and the cleaned collected data is stored in an electronic format prior to analysis of said cleaned collected data.

4. A method according to claim 1, further comprising tracking said composite score over a pre-configured time span.

5. A method according to claim 1, where said composite score further comprises changes in Heart Rate Variability (HRV) data, the biometric data, and data indicating changes in the environment data.

6. A method according to claim 1, further comprising receiving from a user or medical practitioner a threshold composite score or composite score range that is preferred for the user to maintain.

7. A method according to claim 1, further comprising transmitting to a user recommended actions comprising events, interventions, and/or planned steps in accordance with maintaining said user's particular composite score.

8. A method according to claim 1, where said sensors further comprise any of a finger sensor, an LED sensor, a chest-strap electrocardiogram sensor, a camera, or sensors contained within or attached to a mobile device associated with said user.

9. A method according to claim 1, where the composite score is presented to a user as a numeric value and a gauge graphic to permit the user to visually understand changes in the composite score over time.

10. A method according to claim 1, where the composite score, recommendations, and guidance are provided as a report, as part of an ongoing data display, or in real-time as live biofeedback to a user during an activity.

11. A system for biometric monitoring and scoring, comprising:
a data processor having an electronic memory;
receiving biometric data from a plurality of users, said biometric data collected when users are not physically active;
collecting biometric data and physiological data comprising image data, historical health history data, and environmental data associated with said plurality of users over time;
storing all collected and received data in an electronic storage system attached to said data processor;
combining said collected physiological and biometric data to create a combined score that comprises at least heart rate activity data, Heart Rate Variability (HRV) data, and collected biometric data for each of said plurality of users;
comparing said combined score against previously stored combined scores to determine modifications to user actions and activities to permit any of said plurality of users to meet an expressed goal;
presenting HRV-based modifications to user actions and activities in combination with one or more recommended actions related to said modifications to user actions and activities to any of said plurality of users and/or medical service providers associated with any of said plurality of users;
any of said plurality of users implementing said presented HRV-based modifications to user actions and activities to achieve an expressed physiological goal as measured by said composite score.

12. A system according to claim 11, further comprising utilizing one or more signal cleaning algorithms to detect artifacts in the collected data and improve the collected data by removing any detected artifacts that impair the signal quality of the biometric and/or physiological data.

13. A system according to claim 12, where signal cleaning of the collected data is performed during a data collection action and the cleaned collected data is stored in an electronic format prior to analysis of said cleaned collected data.

14. A system according to claim 11, further comprising tracking said composite score over a pre-configured time span.

15. A system according to claim 11, where said composite score further comprises changes in Heart Rate Variability (HRV) data, biometric data, and changes in environment data.

16. A system according to claim 11, further comprising receiving from a user or medical practitioner a threshold composite score or composite score range that is preferred for the user to maintain.

17. A system according to claim 11, further comprising transmitting to a user recommended actions comprising events, interventions, and/or planned steps in accordance with maintaining said user's particular composite score.

18. A system according to claim 11, where said sensors further comprise any of a finger sensor, an LED sensor, a chest-strap electrocardiogram sensor, or sensors contained within or attached to a mobile device associated with said user.

19. A system according to claim 11, where the composite score is presented to a user as a numeric value and a gauge graphic to permit the user to visually understand changes in the composite score over time.

20. A system according to claim 11, where the composite score, recommendations, and guidance are provided as a report, as part of an ongoing data display, or in real-time as live biofeedback to a user during an activity.
